SEQUENCE LISTING (21 sequences)

SEQ ID NO: 1 - protein, 111 aa, synthetic construct
DIVLTQSPATLSVSPGERATISCRASQRVSSSTYSYMHWYQQKPGQPPKLLIKYASNLESGVPARFSGSGSGTDFTLTISSVEPEDFATYYCQHSWEIPPTFGGGTKLEIK

SEQ ID NO: 2 - protein, 118 aa, synthetic construct
QVQLVQSGAEVVKPGASVKLSCKASGYIFTSYYMYWVKQAPGQGLEWIGEINPSNGDTNFNEKFKSKATLTVDKSASTAYMELSSLRSEDTAVYYCTRSDGRNDMDSWGQGTLVTVSS

SEQ ID NO: 3 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 232 aa, synthetic construct
EPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 111 aa, synthetic construct
DIVLTQSPATLSVSPGERATISCRASQKVSSSTYSYMHWYQQKPGQPPKLLIKYASNLESGVPARFSGSGSGTDFTLTISSVEPEDFATYYCQHSWEIPPTFGGGTKLEIK

SEQ ID NO: 6 - protein, 118 aa, synthetic construct
QVQLVQSGAEVVKPGASVKLSCKASGYIFTSYYMYWVKQAPGQGLEWIGEINPSNGDTNFNEKFKSKATLTVDRSASTAYMELSSLRSEDTAVYYCTRSDGRNDMDSWGQGTLVTVSS

SEQ ID NO: 7 - protein, 218 aa, synthetic construct
DIVLTQSPATLSVSPGERATISCRASQRVSSSTYSYMHWYQQKPGQPPKLLIKYASNLESGVPARFSGSGSGTDFTLTISSVEPEDFATYYCQHSWEIPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - DNA, 660 bp, synthetic construct
  source 1..660: mol_type = other DNA
gacatcgtgctgacccagtcccccgccaccctgtccgtgtcccccggcgagagggccaccatctcctgcagggcctcccagagggtgtcctcctccacctactcctacatgcactggtaccagcagaagcccggccagccccccaagctgctgatcaagtacgcctccaacctggagtccggcgtgcccgccaggttctccggctccggctccggcaccgacttcaccctgaccatctcctccgtggagcccgaggacttcgccacctactactgccagcactcctgggagatcccccccaccttcggcggcggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 9 - protein, 448 aa, synthetic construct
QVQLVQSGAEVVKPGASVKLSCKASGYIFTSYYMYWVKQAPGQGLEWIGEINPSNGDTNFNEKFKSKATLTVDKSASTAYMELSSLRSEDTAVYYCTRSDGRNDMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 1956 bp, synthetic construct
  source 1..1956: mol_type = other DNA
caggtgcagctggtgcagtccggcgccgaggtggtgaagcccggcgcctccgtgaagctgtcctgcaaggcctccggctacatcttcacctcctactacatgtactgggtgaagcaggcccccggccagggcctggagtggatcggcgagatcaacccctccaacggcgacaccaacttcaacgagaagttcaagtccaaggccaccctgaccgtggacaagtccgcctccaccgcctacatggagctgtcctccctgaggtccgaggacaccgccgtgtactactgcaccaggtccgacggcaggaacgacatggactcctggggccagggcaccctggtgaccgtgtcctccgctagcaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatctagtgacaaaactcacacaagcccaccgagcccaggtaagccagcccaggcctcgccctccagctcaaggcgggacaggtgccctagagtagcctgcatccagggacaggccccagccgggtgctgacacgtccacctccatctcttcctcagcacctgaactcctggggggatcctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaataatga

SEQ ID NO: 11 - protein, 218 aa, synthetic construct
DIVLTQSPATLSVSPGERATISCRASQKVSSSTYSYMHWYQQKPGQPPKLLIKYASNLESGVPARFSGSGSGTDFTLTISSVEPEDFATYYCQHSWEIPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - DNA, 660 bp, synthetic construct
  source 1..660: mol_type = other DNA
gacatcgtgctgacccagtcccccgccaccctgtccgtgtcccccggcgagagggccaccatctcctgcagggcctcccagaaggtgtcctcctccacctactcctacatgcactggtaccagcagaagcccggccagccccccaagctgctgatcaagtacgcctccaacctggagtccggcgtgcccgccaggttctccggctccggctccggcaccgacttcaccctgaccatctcctccgtggagcccgaggacttcgccacctactactgccagcactcctgggagatcccccccaccttcggcggcggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 13 - protein, 448 aa, synthetic construct
QVQLVQSGAEVVKPGASVKLSCKASGYIFTSYYMYWVKQAPGQGLEWIGEINPSNGDTNFNEKFKSKATLTVDRSASTAYMELSSLRSEDTAVYYCTRSDGRNDMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - DNA, 1956 bp, synthetic construct
  source 1..1956: mol_type = other DNA
caggtgcagctggtgcagtccggcgccgaggtggtgaagcccggcgcctccgtgaagctgtcctgcaaggcctccggctacatcttcacctcctactacatgtactgggtgaagcaggcccccggccagggcctggagtggatcggcgagatcaacccctccaacggcgacaccaacttcaacgagaagttcaagtccaaggccaccctgaccgtggacaggtccgcctccaccgcctacatggagctgtcctccctgaggtccgaggacaccgccgtgtactactgcaccaggtccgacggcaggaacgacatggactcctggggccagggcaccctggtgaccgtgtcctccgctagcaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatctagtgacaaaactcacacaagcccaccgagcccaggtaagccagcccaggcctcgccctccagctcaaggcgggacaggtgccctagagtagcctgcatccagggacaggccccagccgggtgctgacacgtccacctccatctcttcctcagcacctgaactcctggggggatcctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaataatga

SEQ ID NO: 15 - protein, 18 aa, synthetic construct
ISCRASQRVSSSTYSYMH

SEQ ID NO: 16 - protein, 7 aa, synthetic construct
YASNLES

SEQ ID NO: 17 - protein, 9 aa, synthetic construct
QHSWEIPPT

SEQ ID NO: 18 - protein, 5 aa, synthetic construct
SYYMY

SEQ ID NO: 19 - protein, 17 aa, synthetic construct
EINPSNGDTNFNEKFKS

SEQ ID NO: 20 - protein, 9 aa, synthetic construct
SDGRNDMDS

SEQ ID NO: 21 - protein, 448 aa, synthetic construct
QVQLVQSGAEVVKPGASVKLSCKASGYIFTSYYMYWVKQAPGQGLEWIGEINPSNGDTNFNEKFKSKATLTVDKSASTAYMELSSLRSEDTAVYYCTRSDGRNDMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0242493 A1
(43) Pub. Date: Aug. 20, 2026

(54) METHODS FOR THE TREATMENT OF KIDNEY DISEASES OR DISORDERS

(71) Applicant: ELEDON PHARMACEUTICALS, INC., Irvine, CA (US)

(72) Inventor: Steven N. PERRIN, Irvine, CA (US)

(73) Assignee: ELEDON PHARMACEUTICALS, INC., Irvine, CA (US)

(21) Appl. No.: 18/870,289

(22) PCT Filed: May 24, 2023
(86) PCT No.: PCT/US2023/067424

§ 371 Date: Nov. 27, 2024

Related U.S. Application Data
(60) Provisional application No. 63/365,448, filed on May 27, 2022.

(51) Int. Cl. C07K 16/28; A61K 38/17; A61K 39/00; A61K 39/395; A61P 13/12; G01N 33/68

(52) U.S. Cl. CPC C07K 16/2875; A61K 38/1774; A61K 39/3955; A61P 13/12; G01N 33/6854; G01N 33/6863; G01N 33/6893; A61K 2039/505; G01N 2800/347; G01N 2800/52

(57) ABSTRACT

Provided herein are methods and kits for treating kidney-related diseases disorders, and complications, such as Berger's disease and kidney transplant rejections. Also provided are methods of predicting or measuring a response to a treatment by measuring biomarker levels in a sample, and methods of modulating biomarker levels.

### CROSS REFERENCE TO RELATED APPLICATIONS

This application claims priority to U.S. Prov. App. No. 63/365,448, filed May 27, 2022, which is incorporated by reference in its entirety.

### REFERENCE TO SEQUENCE LISTING

The present application is being filed along with a Sequence Listing in electronic format. The Sequence Listing is provided as a file entitled “ELDN.011WO.xml” created on May 1, 2023, which is 27,088 bytes in size. The information in the electronic format of the Sequence Listing is incorporated herein by reference in its entirety.

### BACKGROUND

### Field

The present disclosure relates to methods for treating an autoimmune disorder in a subject, including, for example, a kidney-related autoimmune disorder, such as kidney-related diseases, disorders, or complications, such as Berger's Disease or renal transplantation. Also provided are methods of predicting and/or measuring response to a treatment, including measuring levels of one or more biomarker. Embodiments also relate to methods of modulating a level of one or more biomarker.

### Description of the Related Art

The kidneys are essential organs for filtering blood and regulating the mineral content in a subject. Many diseases and disorders are associated with the kidney, including chronic kidney disease (CKD), diabetic kidney disease, acute kidney injury, kidney failure, polycystic kidney disease, hematuria, Fabry disease, lupus kidney disease, Alport's syndrome, IgM nephropathy, interstitial nephritis, Membranoproliferative Glomerulonephritis, simple kidney cysts, kidney infections, kidney stones, and IgA nephropathy.

IgA nephropathy, also called Berger's Disease or IgAN, is a type of chronic kidney disease. Over approximately 10-20 years, IgA accumulates in a subject's glomeruli and results in the blockade of the filtering system in the kidney. This, in turn, results in the activation of the immune system, and general inflammation around the glomeruli of the kidney. Aggressive IgAN can also affect other major organs, such as the liver, skin, and cardiovascular system.

Severe kidney disease in an individual can result in the need for a kidney organ transplant. However, the individual typically must receive the donated kidney from a donor that ABO blood group and crossmatch (human leukocyte antigen-HLA) compatible. Even with a successful transplant of suitable kidney, the individual must remain on immunosuppressants for the remainder of their lives. Otherwise, their immune runs a high risk of rejecting the new organ. Another potential complication following kidney transplant is graft-versus-host disease, in which the white blood cells of the donor that still remain inside the newly transplanted kidney recognize their new host as foreign and attack the individual's body cells. The high risk of complications following kidney transplant remain a significant barrier in the medical field.

### SUMMARY

Disclosed in this application is a method of predicting the response of a subject with a kidney-related disease or disorder to treatment with a compound against CD40L or CD40. In some embodiments, the method comprises collecting a sample from the subject; and measuring a concentration of at least one biomarker present in the sample, wherein the at least one biomarker is CXCL9, CXCL10, IgM, C3, IgA, IgE, CD40L, and CD40; wherein the concentration of the at least one biomarker is predictive that the subject is likely to be responsive to treatment of the kidney-related disease or disorder with a compound against CD40L or CD40. In some embodiments, the kidney-related disease or disorder comprises inflammation. In some embodiments, the subject is mammalian and/or human. In some embodiments, the compound blocks the interaction between CD40 and CD40L. In some embodiments, the compound is an anti-CD40L antibody or an anti-CD40 antibody. In some embodiments, the compound is AT-1501. In some embodiments, the compound is MR1 or 5c8. In some embodiments, the kidney-related disease or disorder is selected from the group consisting of colitis, drug induced lupus nephritis, lupus nephritis, nephropathy, chronic kidney disease, graft versus host disease, immune graft response, kidney transplant, transplant rejection, atherosclerosis, diabetic kidney disease, acute kidney injury, kidney failure, polycystic kidney disease, hematuria, Fabry disease, lupus kidney disease, Alport's syndrome, IgM nephropathy, interstitial nephritis, Membranoproliferative Glomerulonephritis, simple kidney cysts, kidney infections, kidney stones, and IgA nephropathy. In some embodiments, the kidney-related disease or disorder is an autoimmune disease, wherein the autoimmune disease is systemic lupus erythematous, type-1 diabetes, myasthenia gravis, inflammatory bowel disease, immune thrombocytopenia purpura, rheumatoid arthritis, psoriasis, Addison's disease, Crohn's disease, uveitis, multiple sclerosis, hemolytic anemia, or Graves' disease. In some embodiments, the sample is blood, urine, saliva, or mucus. In some embodiments, the sample is urine.

Also disclosed herein is a method of treating a subject with a kidney-related disease or disorder. In some embodiments, the method comprises administering a therapeutically effective dose of a compound against CD40L or CD40. In some embodiments, the kidney-related disease or disorder comprises inflammation. In some embodiments, the subject is mammalian and/or human. In some embodiments, the compound blocks the interaction between CD40 and CD40L. In some embodiments, the compound is an anti-CD40L antibody or an anti-CD40 antibody. In some embodiments, the compound is AT-1501. In some embodiments, the compound is MR1 or 5c8. In some embodiments, the compound is administered at least once every three weeks. In some embodiments, the compound is administered at a dose of between 200 mg/kg and 100 mg/kg; preferably at a dose between 1 mg/kg and 20 mg/kg. In some embodiments, the kidney-related disease or disorder is colitis, drug induced lupus nephritis, lupus nephritis, nephropathy, chronic kidney disease, graft versus host disease, immune graft response, transplant rejection, atherosclerosis, diabetic kidney disease, acute kidney injury, kidney failure, polycystic kidney disease, hematuria, Fabry disease, lupus kidney disease, Alport's syndrome, IgM nephropathy, interstitial nephritis, Membranoproliferative Glomerulonephritis, simple kidney cysts, kidney infections, kidney stones, or IgA nephropathy. In some embodiments, the kidney-related disease or disorder is an autoimmune disease, wherein the autoimmune disease is systemic lupus erythematous, type-1 diabetes, myasthenia gravis, inflammatory bowel disease, immune thrombocytopenia purpura, rheumatoid arthritis, psoriasis, Addison's disease, Crohn's disease, uveitis, multiple sclerosis, hemolytic anemia, or Graves' disease. In some embodiments, the method further comprises administering a second pharmaceutically effective compound. In some embodiments, the second compound blocks the interaction between CD28 and CD86 or between CD28 and CD80. In some embodiments, the second compound targets at least one of the biomarkers selected from the group consisting of: interleukins, cytokines, and proinflammatory markers. In some embodiments, the second compound is a CTLA4-Ig fusion protein, an abatacept, a belatacept, or a galiximab. In some embodiments, the compound is administered orally, parenterally, or topically. In some embodiments, the compound is administered parenterally. In some embodiments, the compound is administered by injection, most preferably intravenous, intramuscular, or subcutaneous injection. In some embodiments, the compound that targets CD40L or CD40 is an antibody that comprises: a heavy chain variable region (V_{H}) comprising: (1) a CDRH1 domain comprising the sequence set forth in SEQ ID NO: 9; (2) a CDRH2 domain comprising the sequence set forth in SEQ ID NO: 10, 11, 12, 13, or 14; and (3) a CDRH3 domain comprising the sequence set forth in SEQ ID NO: 15; and also comprises a light chain variable region (V_{L}) comprising: (4) a CDRL1 domain comprising the sequence set forth in SEQ ID NO: 16 or 17; (5) a CDRL2 domain comprising the sequence set forth in SEQ ID NO: 18 or 19; and (6) a CDRL3 domain comprising the sequence set forth in SEQ ID NO: 20. In some embodiments, the compound that targets CD40L or CD40 is an antibody that comprises: a heavy chain variable region (V_{H}) having the amino acid sequence as set forth in SEQ ID NOs: 1, 2, 3, or 4; and a light chain variable region (V_{L}) having the amino acid sequence as set forth in SEQ ID NOs: 5, 6, 7, or 8.

Also disclosed herein is a method of modulating the concentration of at least one biomarker in a subject. In some embodiments, the method comprises administering to the subject a therapeutically effective amount of a compound against CD40L or CD40, wherein the biomarkers are selected from the group consisting of: IgA, IgE, CD40L, CD40, C3, CXCL9, CXCL10, and IgM. In some embodiments, the concentration of at least one biomarker increases following administration of the compound. In some embodiments, the concentration of at least one biomarker decreases following administration of the compound. In some embodiments, the concentration of at least one biomarker increases, and the concentration of at least one other biomarker decreases, following administration of the compound. In some embodiments, the subject has a kidney-related disease or disorder. In some embodiments, the kidney-related disease or disorder comprises inflammation. In some embodiments, the subject is mammalian and/or human. In some embodiments, the compound blocks the interaction between CD40 and CD40L. In some embodiments, the compound is an anti-CD40L antibody or an anti-CD40 antibody. In some embodiments, the compound is AT-1501. In some embodiments, the compound is MR1 or 5c8. In some embodiments, the kidney-related disease or disorder is colitis, drug induced lupus nephritis, lupus nephritis, nephropathy, chronic kidney disease, graft versus host disease, immune graft response, transplant rejection, atherosclerosis, diabetic kidney disease, acute kidney injury, kidney failure, polycystic kidney disease, hematuria, Fabry disease, lupus kidney disease, Alport's syndrome, IgM nephropathy, interstitial nephritis, Membranoproliferative Glomerulonephritis, simple kidney cysts, kidney infections, kidney stones, or IgA nephropathy. In some embodiments, the kidney-related disease or disorder is an autoimmune disease, wherein the autoimmune disease is systemic lupus erythematous, type-1 diabetes, myasthenia gravis, inflammatory bowel disease, immune thrombocytopenia purpura, rheumatoid arthritis, psoriasis, Addison's disease, Crohn's disease, uveitis, multiple sclerosis, hemolytic anemia, or Graves' disease. In some embodiments, the compound is administered at least once every three weeks. In some embodiments, the compound is administered at a dose of between 200 mg/kg and 100 mg/kg; preferably at a dose between 1 mg/kg and 20 mg/kg. In some embodiments, the compound is administered orally, parenterally, or topically. In some embodiments, the compound is administered parenterally. In some embodiments, the compound is administered by injection, most preferably intravenous, intramuscular, or subcutaneous injection. In some embodiments, the compound that targets CD40L or CD40 is an antibody that comprises a heavy chain variable region (V_{H}) comprising: (1) a CDRH1 domain comprising the sequence set forth in SEQ ID NO: 9; (2) a CDRH2 domain comprising the sequence set forth in SEQ ID NO: 10, 11, 12, 13, or 14; and (3) a CDRH3 domain comprising the sequence set forth in SEQ ID NO: 15; and comprises a light chain variable region (V_{L}) comprising: (4) a CDRL1 domain comprising the sequence set forth in SEQ ID NO: 16 or 17; (5) a CDRL2 domain comprising the sequence set forth in SEQ ID NO: 18 or 19; and (6) a CDRL3 domain comprising the sequence set forth in SEQ ID NO: 20. In some embodiments, the compound that targets CD40L or CD40 is an antibody that comprises: a heavy chain variable region (V_{H}) having the amino acid sequence as set forth in SEQ ID NOs: 1, 2, 3, or 4; and a light chain variable region (V_{L}) having the amino acid sequence as set forth in SEQ ID NOs: 5, 6, 7, or 8.

### BRIEF DESCRIPTION OF THE DRAWINGS

FIG. 1A shows MR1 concentration over time using a linear concentration (Y) axis.

FIG. 1B shows MR1 concentration over time using a log concentration (Y) axis.

FIG. 2 is a graph of the mean daily body weight measurements starting at day 40 to attainment of peak body weight for the control and the treatment group.

FIG. 3 depicts the mean daily body weight measurements from peak body weight to death for the control and treatment group.

FIG. 4 shows a Kaplan Meier Survival Plot for the control and treatment group as calculated by a Cox proportional hazard model.

FIG. 5A is a graph of the mean daily body weight measurements starting at day 40 to attainment of peak body weight for the control group and the MR1 treated group.

FIG. 5B is a graph depicting the mean daily body weight measurements from peak body weight to death for the control group and the MR1 treated group.

FIG. 5C shows the time to disease onset based on the time to progress to a neurological score of 2 in the control group and the MR1 treated group.

FIG. 5D shows the mean daily body weight measurements from peak body weight to death for the control and the MR1 treated group.

FIG. 6 is a graph showing the relative binding to human CD40L, of JB5 antibody (circles, dotted line), hu5c8 antibody (squares-solid line), and the control CTLA4-IgG1 (triangles).

FIG. 7 provides the amino acid sequences for the hu5c8 and JB5 VL region (SEQ ID NO: 1), hu5c8 and JB5 VH region (SEQ ID NO: 2), the Fc region (SEQ ID NO: 3), the JB5 Fc region (SEQ ID NO: 4), the JB5-R28K VL region (SEQ ID NO: 5), the JB5-K74R VH region (SEQ ID NO: 6), and the JB5 light chain Amino acid sequence (SEQ ID NO: 7).

FIG. 8 provides the light chain synthetic nucleotide sequence that encodes the anti-CD40L antibody JB5 (SEQ ID NO: 8), upper case letters represent the exons and the lower case letters represent the intron sequences of the synthetic gene, and also provides the heavy chain amino acid sequence of the anti-CD40L antibody JB5 (SEQ ID NO: 9).

FIG. 9 provides a synthetic nucleic acid sequence that encodes the heavy chain of the anti-CD40L antibody JB5 (SEQ ID NO: 10), upper case letters represent the exons, and the lower-case letters represent the intron sequences of the synthetic gene.

FIG. 10 provides the amino acid sequence of the anti-CD40L antibody JB5-R28K (SEQ ID NO: 11), a synthetic nucleic acid sequence that encodes the light chain of the anti-CD40L antibody JB5-R28K (SEQ ID NO: 12), upper case letters represent the exons and the lower case letters represent the intron sequences of the synthetic gene, and also provides the heavy chain amino acid sequence of the anti-CD40L antibody JB5-K74R (SEQ ID NO: 13).

FIG. 11 provides a synthetic nucleic acid sequence that encodes the heavy chain of the anti-CD40L antibody JB5-K74R (SEQ ID NO: 14) upper case letters represent the exons, and the lower-case letters represent the intron sequences of the synthetic gene.

FIG. 12 provides the amino acid sequences of the CDRs of the heavy and light chain of the anti-CD40L antibody JB5 (SEQ ID NOs: 15-20, respectively) and the amino acid sequence of the hu5C8 heavy chain (SEQ ID NO: 21).

FIG. 13A is a bar graph showing the ranked potency of 16 antibody clones versus the anti-CD40L antibody 5c8. Ranked potency is IC50 clone/IC50 of 5c8×100.

FIG. 13B is a bar graph showing the ranked potency of 16 antibody clones versus the anti-CD40L antibody AT-1501. Ranked potency is IC50 clone/IC50 of AT-1501×100.

FIGS. 14A-14G and 14I-14Q show the binding curves of the antibodies from each of the clones with the binding curve for 5c8. FIG. 14H shows the binding curves of AT-1501 and 5c8.

FIGS. 15A-15G and 151-15Q show the binding curves of the antibodies from each of the clones with the binding curve for AT-1501. FIG. 15H shows the binding curves of AT-1501 and 5c8.

FIGS. 16A and 16B are graphs, each of which shows binding of eight of the antibody clones, 5c8, AT-1501 and abatacept to FcγRI. The only antibody having significant binding is the 5c8 antibody.

FIGS. 17A and 17B are graphs, each of which shows binding of eight of the antibody clones, 5c8, AT-1501 and abatacept to FcγRIIa. The only antibody having significant binding is the 5c8 antibody.

FIGS. 18A and 18B are graphs, each of which shows binding of eight of the antibody clones, 5c8, AT-1501 and abatacept to FcγRIIIa. None of the antibodies showed significant binding to FcγRIIIa.

FIGS. 18C and 18D are graphs, each of which shows binding of eight of the antibody clones, 5c8, AT-1501 and abatacept to FcγRIIIb. None of the antibodies showed significant binding to FcγRIIIb.

FIGS. 19A and 19B are graphs, each showing the binding of eight of the antibody clones, 5c8, AT-1501 and abatacept to C1q. The only antibody having significant binding to C1q was the 5c8 antibody.

FIGS. 20A-20D are graphs for the change in IgAN-associated proinflammatory markers in human subjects, 12 weeks after kidney transplants. An “*” represents data with a p value between 0.05 and 0.01; an “**” represents data with a p value between 0.01 and 0.001; an “****” represents data with a p value that is less than 0.001. The data plotted are for the IgAN-associated proinflammatory markers IgA (FIG. 20A), IgE (FIG. 20B), CD40L (FIG. 20C), and C3 (FIG. 20D).

FIGS. 21A-21D are graphs for the change in general transplant proinflammatory markers in human subjects, 12 weeks after kidney transplants. An “*” represents data with a p value between 0.05 and 0.01; an “**” represents data with a p value between 0.01 and 0.001; an “***” represents data with a p value that is less than 0.001. The data plotted are for the general transplant-associated proinflammatory markers CXCL9 (FIG. 21A), CXCL10 (FIG. 21B), IgM (FIG. 21C), and C3 (FIG. 21D).

FIG. 22 depicts a non-limiting example schematic of the study design and data output for Phase 1b and 2 clinical trials, as discussed in Example 2.

FIG. 23 depicts the demographics of the first four participants used in the Phase 1b clinical trial, as discussed in Example 2.

FIG. 24 depicts a non-limiting example of changes in kidney function (as monitored through eGFR levels) over time in patients following kidney transplants and treatment, as discussed in Example 2.

FIG. 25 depicts a non-limiting example distribution of eGFR levels and cox proportional hazard model of graft loss across human participants, as discussed in Example 2.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein. All references cited herein are expressly incorporated by reference herein in their entirety and for the specific disclosure referenced herein.

The present disclosure describes methods of treating a patient having an autoimmune disorder, such as an autoimmune disorder associated with the kidney, including, for example, a kidney disease, disorder, or complication by administering a therapeutically effective amount of a compound that blocks the interaction of CD40 and CD40 ligand (CD40L). The disclosure also describes methods of treating a patient by co-administering a compound that blocks the interaction of CD40 and CD40L with a compound that blocks the interaction between CD28 and CD86 or between CD28 and CD80.

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there is a plurality of definitions for a term herein, those in this section prevail unless stated otherwise.

One of ordinary skill in the art will appreciate that starting materials, biological and chemical materials, biological and chemical reagents, synthetic methods, purification methods, analytical methods, assay methods, and biological methods other than those specifically exemplified can be employed in the practice of the disclosure without resort to undue experimentation. All art-known functional equivalents, of any such materials and methods are intended to be included in this disclosure.

As used herein, “a” or “an” can mean one or more than one.

As used herein, the term “about” or “approximately” has its usual meaning as understood by those skilled in the art and thus indicates that a value includes the inherent variation of error for the method being employed to determine a value, or the variation that exists among multiple determinations.

The use of the conjunction “or” is used interchangeably with at “least one of”. For example: where a composition comprises A or B, the method must comprise at least one of A and B but may also comprise both A and B. Likewise a composition comprising “A, B, C or D” must comprise at least one of the group of A, B, C and D, but may also comprise all or any combination of A, B, C and D.

Amino acid substitutions are denoted by the convention in which the original amino acid, the position of the amino acid in the specified sequence and the replacement amino acid are identified, for example, C11S would indicate that the cysteine at position 11 of the polypeptide sequence is replaced with a serine.

Humanized antibodies are antibodies produced from non-human species whose protein sequences have been modified to increase their similarity to antibody variants produced naturally in humans. The process of “humanization” is usually applied to monoclonal antibodies developed for administration to humans (for example, antibodies developed as anti-cancer drugs).

Currently it is common to humanize a non-human antibody by insertion of relevant CDRs from antibodies created in a non-human animal into a human antibody “scaffold”. “Direct” creation of a humanized antibody can be accomplished by inserting the appropriate CDR coding segments (responsible for the desired binding properties) into a human antibody “scaffold.” This may be achieved through recombinant DNA methods using an appropriate vector and expression in mammalian cells. That is, after an antibody is developed to have the desired properties in a mouse (or other non-human), the DNA coding for that antibody can be isolated, cloned into a vector and sequenced. The DNA sequence corresponding to the antibody CDRs can then be determined. Once the precise sequence of the desired CDRs is known, a strategy can be devised for inserting these sequences appropriately into a construct containing the DNA for a human antibody variant. The CDRs may also be varied, e.g., to increase specificity, prior to insertion into the scaffold.

The term human antibody refers to an antibody which possesses an amino acid sequence which corresponds to that of an antibody produced by a human and/or has been made using any technique for making human antibodies. This definition of a human antibody specifically excludes a humanized antibody comprising non-human antigen binding residues. The term “variant” as used herein is defined as a modified or altered form of a wildtype sequence, e.g., where one or more amino acids may be replaced by other amino acid(s) or non-amino acid(s) which do not substantially affect function. In some embodiments, the variant may contain an altered side chain for at least one amino acid residue.

The term “antigen” as used herein is defined as an entity that can stimulate the production of antibodies and specifically combine with them and/or an entity which elicits an immune system response. For example, a cell surface protein or a specific linear or non-linear portion thereof. The term herein may be abbreviated to “Ag.”

An “antigen binding antibody fragment” refers to a molecule other than an intact antibody that comprises a portion of an intact antibody that binds the antigen to which the intact antibody binds. Examples of antibody fragments include but are not limited to Fv, Fab, Fab′, Fd fragments, dAb fragments, Fab′-SH, F(ab′) 2; diabodies; triabodies; linear antibodies; single-chain antibody molecules (e.g., scFv); and multispecific antibodies formed from antibody fragments, minimal recognition units consisting of the amino acid residues that mimic the hypervariable region of an antibody (e.g., an isolated complementarity determining region (CDR) such as a CDR3 peptide). An antigen binding fragment as disclosed in the present application binds to the antigen CD40L.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

The isolated antibody includes bispecific antibodies in which each arm of the antibody or the antigen binding fragment binds to a different target or epitope.

The terms “full length antibody,” “intact antibody,” and “whole antibody” are used herein interchangeably to refer to an antibody having a structure substantially similar to a native antibody structure or having heavy chains that contain an Fc region.

“5c8” refers to the mouse anti-human antibody that binds CD40L and is produced by the hybridoma that is available from the ATCC having the accession number HB10916 and is described in U.S. Pat. No. 5,474,771. “hu5c8” refers to a humanized version of 5c8 the sequence of which is disclosed in Karpusas, et al., Structure vol. 9, pp 321-329, (2001).

The term “specifically binds,” or the like, means that an antibody or antigen-binding fragment thereof forms a complex with an antigen that is relatively stable under physiological conditions. Specific binding may be characterized by an equilibrium dissociation constant (KD) of about 3000 nM or less (i.e., a smaller KD denotes a tighter binding), about 2000 nM or less, about 1000 nM or less; about 500 nM or less; about 300 nM or less; about 200 nM or less; about 100 nM or less; about 50 nM or less; about 1 nM or less; or about 0.5 nM. Specific binding for a particular antigen or an epitope may be exhibited, for example, by an antibody having a KD for an antigen or epitope of at least about 1×10⁻⁴ M, at least about 1×10⁻⁵ M, at least about 1×10⁻⁶ M, at least about 1×10⁻⁷ M, at least about 1×10⁻⁸ M, at least about 1×10⁻⁹ M, alternatively at least about 1×10⁻¹⁰ M, at least about 1×10⁻¹¹ M, at least about 1×10⁻¹² M, or greater, where KD refers to a equilibrium dissociation constant of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope. Also, specific binding for a particular antigen or an epitope may be exhibited, for example, by an antibody having a Ka for an antigen or epitope of at least 20-, 50-, 100-, 500-, 1000-, 5,000-, 10,000- or more times greater for the epitope relative to a control, where Ka refers to an association rate of a particular antibody-antigen interaction.

The term “neutralizing antibody” includes an antibody that is capable of inhibiting and/or neutralizing the biological activity of CD40L, for example an anti-CD40L antibody or antigen fragment thereof that inhibits or prevents or diminishes the binding of CD40L to CD40, and thus inhibiting or reducing the signaling pathway triggered by CD40L and/or inhibiting or reducing the binding of CD40L to CD40.

The terms “antagonistic antibody” or “antagonist antibody” are used herein equivalently and include an antibody that is capable of inhibiting and/or neutralizing the biological signaling activity of CD40L, as described for a neutralizing antibody supra.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the methods and compositions disclosed herein. Thus, it should be understood that although aspects of the present disclosure have been specifically disclosed by various embodiments which may include preferred embodiments, exemplary embodiments and optional features, modifications and variations of the concepts herein disclosed may be resorted to by those skilled in the art. Such modifications and variations are considered to be within the scope of embodiments of the disclosure as described and as may be defined by the appended claims.

Reference in the specification is made to percent identity between polypeptide or amino acid sequences. The percent identity between the two sequences is a function of the number of identical positions shared by the sequences, taking into account the number of gaps, and the length of each gap, which need to be introduced for optimal alignment of the two sequences. Identity can be measured as “local identity” or “global identity.” Local identity refers the degree of sequence relatedness between polypeptides as determined by the match between strings of such sequences. Global identity refers to the degree of sequence relatedness of a polypeptide compared to the full-length of a reference polypeptide. Unless specified otherwise, as used herein, identity means global identity. For the purposes of this disclosure, the percentages for global identity are calculated using Needleman and Wunsch ((1970) J. Mol. Biol. 48:444-453) algorithm using a Blossum 62 scoring matrix with a gap penalty of 12, a gap extend penalty of 4, and a frameshift gap penalty of 5. There are many publicly available software programs that incorporate the Needleman and Wunsch algorithm, e.g., the GAP program in the GCG software package.

CD40L is also known as CD154, gp39, T-BAM, 5c8 antigen, or TNF related activation protein (TRAP).

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “function” and “functional” as used herein have their plain and ordinary meaning as understood in light of the specification, and refer to a biological, enzymatic, or therapeutic function.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 81, 82, 83, 84, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

The term “isolated protein” or “isolated polypeptide” (e.g., an isolated antibody or isolated antigen binding fragment) is a protein or polypeptide that by virtue of its origin or source of derivation is not associated with naturally associated components that accompany it in its native state; is substantially free of other proteins from the same species; is expressed by a cell from a different species; or does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

As used herein, “in vivo” is given its plain and ordinary meaning as understood in light of the specification and refers to the performance of a method inside living organisms, usually animals, mammals, including humans, and plants, or living cells which make up these living organisms, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

As used herein, “nucleic acid”, “nucleic acid molecule”, or “nucleotide” refers to polynucleotides or oligonucleotides such as deoxyribonucleic acid (DNA) or ribonucleic acid (RNA), oligonucleotides, fragments generated by the polymerase chain reaction (PCR), and fragments generated by any of ligation, scission, endonuclease action, exonuclease action, and by synthetic generation. Nucleic acid molecules can be composed of monomers that are naturally occurring nucleotides (such as DNA and RNA), or analogs of naturally occurring nucleotides (e.g., enantiomeric forms of naturally occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally occurring or modified nucleic acid bases attached to a polyamide backbone. Nucleic acids can be either single stranded or double stranded.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

The term “gene” as used herein have their plain and ordinary meaning as understood in light of the specification, and generally refers to a portion of a nucleic acid that encodes a protein or functional RNA; however, the term may optionally encompass regulatory sequences. It will be appreciated by those of ordinary skill in the art that the term “gene” may include gene regulatory sequences (e.g., promoters, enhancers, etc.) and/or intron sequences. It will further be appreciated that definitions of gene include references to nucleic acids that do not encode proteins but rather encode functional RNA molecules such as tRNAs and miRNAs. In some cases, the gene includes regulatory sequences involved in transcription, or message production or composition. In other embodiments, the gene comprises transcribed sequences that encode for a protein, polypeptide, or peptide. In keeping with the terminology described herein, an “isolated gene” may comprise transcribed nucleic acid(s), regulatory sequences, coding sequences, or the like, isolated substantially away from other such sequences, such as other naturally occurring genes, regulatory sequences, polypeptide, or peptide encoding sequences, etc. In this respect, the term “gene” is used for simplicity to refer to a nucleic acid comprising a nucleotide sequence that is transcribed, and the complement thereof. As will be understood by those in the art, this functional term “gene” includes both genomic sequences, RNA or cDNA sequences, or smaller engineered nucleic acid segments, including nucleic acid segments of a non-transcribed part of a gene, including but not limited to the non-transcribed promoter or enhancer regions of a gene. Smaller engineered gene nucleic acid segments may express or may be adapted to express using nucleic acid manipulation technology, proteins, polypeptides, domains, peptides, fusion proteins, mutants and/or such like.

The terms “individual”, “subject”, “host,” or “patient” as used herein have their usual meaning as understood by those skilled in the art and thus includes a human or a non-human mammal. The term “mammal” is used in its usual biological sense. Thus, it specifically includes, but is not limited to, primates, including simians (chimpanzees, apes, monkeys), humans, cattle, horses, sheep, goats, swine, rabbits, dogs, cats, rodents, rats, mice, or guinea pigs.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm”.

The following abbreviations are used herein: super oxide dismutases-1 (SOD1); T cell receptor (TCR); major histocompatibility complex (MHC) antigen presenting cell (APC); Phosphate buffered saline (PBS), complementarity determining regions (CDR). “IP” means intraperitoneally and “IV” means intravenously.

R1 is a hamster monoclonal antibody that binds to mouse CD40 ligand, “Wild type” as used herein means a non-transgenic mouse. As used herein “small molecule” means a compound having a molecular weight of less than 2000 Daltons. “Treatment” or “treating” as used herein includes prophylactic and therapeutic treatment. The phrase “therapeutically effective amount” means an amount of a compound, or a pharmaceutically acceptable salt thereof, sufficient to inhibit, or allow an improvement in the disorder or condition being treated with administered alone or in conjunction with another pharmaceutical agent or treatment in a particular subject or subject population.

An “effective amount” of an agent, e.g., a pharmaceutical formulation, refers to an amount effective, at dosages and for periods of time necessary, to achieve the desired therapeutic or prophylactic result.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y may generally be represented by the equilibrium dissociation constant (KD), a ratio of k_{off}/kₒₙ, between the antibody and its antigen. KD and affinity are inversely related. The KD value relates to the concentration of antibody (the amount of antibody needed for a particular experiment) and so the lower the KD value (lower concentration) and thus the higher the affinity of the antibody. Affinity may be measured by common methods known in the art, including those described herein. Specific, illustrative, and exemplary embodiments for measuring binding affinity may be measured by radioimmunoassays (RIA), Surface Plasmon Resonance (SPR) on a BIAcore® instrument (GE Healthcare Europe GmbH, Glattbrugg, Switzerland) by capturing the antibody on a protein-A coupled CM5 research grade sensor chip (GE Healthcare Europe GmbH, Glattbrugg, Switzerland; BR-1000-14) with a human CD40 ligand polypeptide used as analyte. Other methods may include radioimmunoassays, and the Kinetic Exclusion Assay. The Kinetic Exclusion Assay is a general-purpose immunoassay platform that is capable of measuring equilibrium dissociation constants, and association and dissociation rate constants for antigen/anti-body interactions.

TMB is an abbreviation of 3,3′,5,5′-Tetramethylbenzidine.

“CDR domain” as used herein means an antibody complementary determining region with or without flanking sequences.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation. One effector function is ability of the Fc or constant region of an antibody to bind proteins including, but not limited to, an Fc receptor (FcR) (e.g., high affinity Fc region of IgG receptor Ia (FCγRIa) (CD64), low affinity immunoglobulin gamma Fc region acceptor IIa (FCγRIIa) (CD32), low affinity immunoglobulin gamma Fc region receptor IIIa (FCγRIIIA) (CD16a), low affinity immunoglobulin gamma Fc region receptor IIIb (FCγRIIIb) (CD16b)). In some embodiments, where the antibodies and antigen binding fragments thereof, have an Fc domain, the Fc domain has been engineered to reduce or eliminate one or more Fc effector function. In a preferred embodiment the Fc domain has been engineered to reduce or eliminate platelet activation and/or platelet aggregation and the concomitant risk of thromboembolism.

### Identification of Candidate Compounds

The co-stimulatory pathway involves, along with other interactions, the binding of CD40 on B cells to CD40L (also known as CD154, gp39, T-BAM, 5c8 antigen, CD40CR and TRAP) on T cells. Human CD40 is expressed on mature B cells, as well as macrophages, dendritic cells, fibroblasts and activated endothelial cells. It is thought that blockade of the CD40:CD40L binding promotes the development of Type I T-helper cell responses.

Using compounds that block these interactions and inhibit the co-stimulatory signals, a significant body of work has demonstrated the immunomodulatory effects of blocking one or more of CD40L, CD80 or CD86 in preclinical models of transplantation and autoimmunity. Blocking CD40L function with blocking antibodies or adenoviral expression of CD40L-Ig improves allograft transplant by 30 to 90 days. Similar studies blocking CD80/CD86 on APCs with CTLA4-Ig or adenoviral expression of CTLA4-Ig transiently improves allograft transplant survival. Transplant rejection in these models is transient and graft rejection ensues over time. Longer term repression of transplant rejection can be accomplished by blocking both the co-stimulatory pathway with CTLA4-Ig and blocking CD40L activation of APCs with anti-CD40L antibodies.

Blocking antibodies to CD40L or genetic deletion of CD40L in mice has demonstrated that CD40L ameliorates disease progression, survival, and surrogate markers of disease in preclinical models of experimental allergic encephalomyelitis (EAE) a model of multiple sclerosis, collagen induced arthritis, and systemic lupus. Blockade of CD40:CD40L binding appears to reduce the ability of macrophages to produce nitric oxide, which mediates many of the macrophages' pro-inflammatory activities.

It appears from such studies that blocking CD40:CD40L interactions and/or blocking of CD28:CD80 or CD28:CD86 interactions can modulate immune responses.

Immunohistochemical data was shown to correlate well with the gene expression data and these data identify macrophages as an antigen presenting cell infiltrating skeletal muscle during disease progression in the G93A mouse.

In order to determine the presence and localization of potential antigen presenting cells in gastrocnemius muscle from G93A and wild type animals, immunohistochemistry was performed on gastrocnemius tissues that were harvested from G93A and wildtype mice, at day 110. Immediately after harvesting, the tissues were embedded in OCT. Frozen sections were H&E stained and hybridized with antibodies to myelin (anti-S100b antibody) or antibodies to hematopoietic cell lineages including T cells (anti-CD3 antibody), B cells (CD45R pan B cell antibody), and macrophages (anti-CD11b antibody). At day 110 there was infiltration of CD11b positive macrophages, and the macrophages appear to be localized to the axons of nerves innervating the skeletal muscle. The localization of macrophages is not dispersed across the entire muscle suggesting that the inflammation is not due to muscle atrophy or muscle fiber remodeling.

In order to confirm that identity of the monocyte lineage cells in the skeletal muscle at day 110 and to clarify the relevance of these cells compared to non-transgenic animals, additional immunohistochemistry was performed with a panel of antibodies specific for the macrophage lineage. An anti-S100b antibody was utilized to label the myelin associated axons innervating the skeletal muscle. All of the macrophage specific antibodies (anti-CD11b antibody, anti-CD86 antibody and anti-MAC1antibody) localized macrophages to the axons of nerves innervating the skeletal muscle of G93A mice with no macrophages present on the axons of wildtype animals. The localization of macrophages was specific for nerves innervating the muscle with no macrophages present on myofibers in the muscle.

The gene expression data suggest that the genes associated with the co-stimulatory pathway are temporally increasing during disease progression both in spinal cord as well as in skeletal muscle. In order to characterize the timing of macrophage infiltration into skeletal muscle immunohistochemistry was performed on gastrocnemius sections from days 60, 80, and 100 in G93A mice. There was no evidence of macrophage infiltration and localization to axons at day 60. Macrophages were evident at day 80 and localized to the axons innervating the muscle as described previously for skeletal muscle at day 110. The number of macrophages increased between day 80 and day 100 and accumulation of macrophages was specific to the axons innervating the skeletal muscle.

In order to quantitate the increase in macrophage infiltration, representative sections from 5 G93A and 5 wild type animals were hybridized with anti-CD86 antibody and the number of macrophages per 10,000 square microns were counted. The number of macrophages in the wild type animals was the same at the 60-, 80- and 110-day time points. Macrophages accumulate in skeletal muscle temporally between days 80 and 100 and there are very few macrophages present in wild type skeletal muscle.

In summary, the immunohistochemical data correlates very well with the gene expression data and identify macrophages as the antigen presenting cell infiltrating skeletal muscle during disease progression in the G93A mouse model. An unexpected finding is that the macrophage infiltration appears to be specifically targeted to the axons innervating the skeletal muscle as localized by labeling with antibodies to myelin and macrophages.

Tissue levels of MR1 directed against murine CD40L were determined using a matrix matched, non-competitive enzyme linked immunosorbent assay (ELISA) in the sandwich format. Seven-point standard curves were included on each plate. Standards were prepared using purified MR1 spiked into PBS diluent solution. PBS diluent solution was matrix matched with the normal mouse tissue at the equivalent dilution of the unknown samples to correct for any non-specific effects resulting from tissue lysates.

84 plasma samples were taken for pharmacokinetic analysis over a period of two weeks after dosing (10 mg/kg, IP) in both female and male G93A mice.

The elimination half-life was similar in females (23 d) and males (22 d) and similar to the half-life for a typical mouse IgG2-based antibody in the mouse. No signs of anti-hamster antibody response were seen.

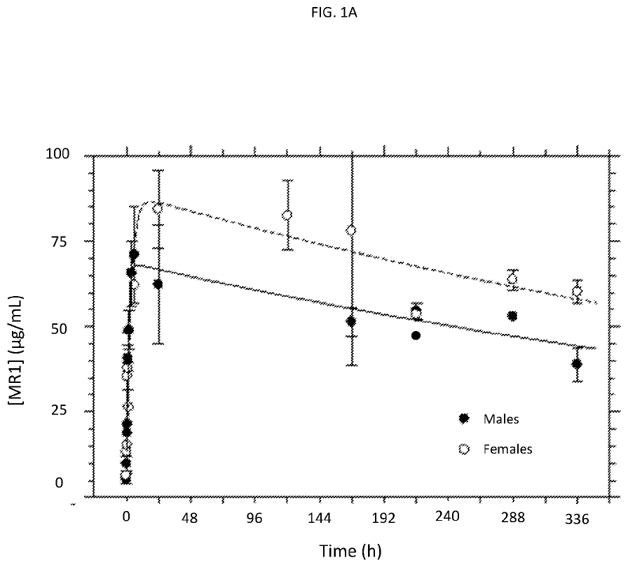

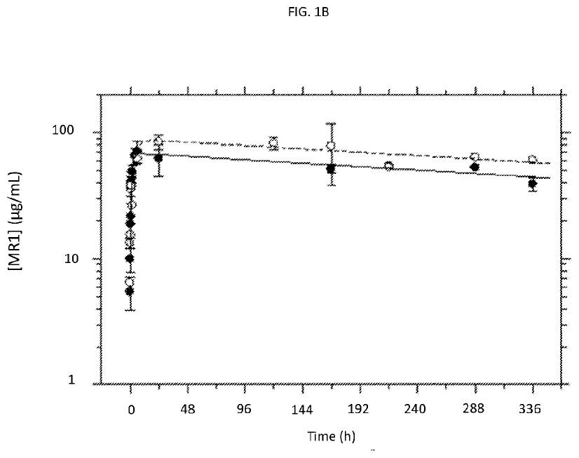

Females show a somewhat smaller volume of distribution for MR1 than males, and thus show higher plasma levels when given the same 10 mg/kg dose. Males show a faster clearance and higher volume of distribution. Thus, to attain similar plasma levels, males would require a higher dose. FIG. 1A shows MR1 concentration over time using a linear concentration (Y) axis. FIG. 1B shows MR1 concentration over time using a log concentration (Y) axis.

36 female G93A mice were litter matched and randomized into two study groups. 18 G93A mice were placed into the MR1-treatment group and the other 18 G93A mice were placed into the control group. Study days are based on days from birth.

A single injection of 56 ug of MR1 was administered intraperitoneally (IP) on day 50. Subsequent to the bolus injection a weekly maintenance injection of 18 ug of MR1 was administered by IP injection. Doses were prepared in vehicle (phosphate buffered saline (PBS, pH 7.3)), to a total volume 200 μl. Control animals where administered 200 μl PBS. Beginning at day 54, animals were monitored daily throughout the course of the study and daily body weight measurements were taken.

Mutant SOD1 transgenic animals display normal body weight (BW) characteristics as neonates and gain weight normally compared to non-transgenic animals into adulthood. Depending on the nature of the genetic mutation in the transgene and the number of copies of mutant transgene weight loss becomes apparent in adult animals and continues until death. Analysis of weight loss in treatment and control groups can provide insight into putative treatment effects on disease onset and rate of progression. In order to assess the impact of MR1 treatment on body weight, two summary parameters are examined (1) Changes in BW from initiation of study to the attainment of peak body weight which may reflect an impact on disease onset (2) The changes in BW from peak body weight until death which may reflect an impact on disease progression.

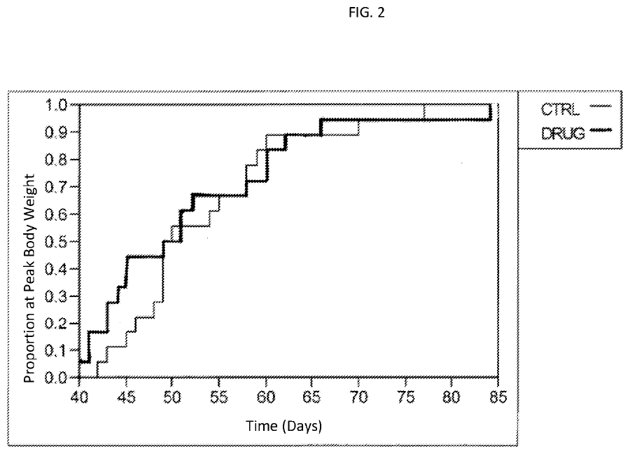

Comparative MR1-treated and control group time-to-event curves for the time from day 40 to the attainment of peak body weight are shown in FIG. 2. The median time to peak body weight for the control group was 50 days compared to the MR1 treated group which was 51 days. This difference was not significant when analyzed by Kaplan Meier using the log rank and Wilcoxon statistical models, Cox proportional hazard, or parametric statistical tests.

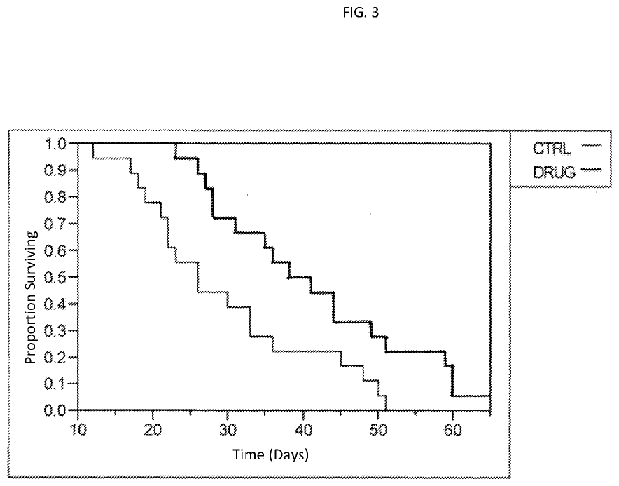

Comparative MR1-treated and control group time-to-event curves for the time from peak body weight to death are shown in FIG. 3. Time from peak body weight to death was statistically significantly later by 15 days in MR1 treated animals. Control animals have a 2.4 to 4.7-fold greater risk of dying sooner after attaining peak body weight than did MR1-treated animals. The median time from peak body weight to death in the control group was 26 days whereas in the MR1 treated group it was 41 days. Significance for each of the analyses in this example was calculated in several ways in order to better evaluate significance. The delay is statistically significant when analyzed using several approaches. (Kaplan Meier, log rank p=0.0110 and Wilcoxon, p=0.0069; Cox proportional Hazard model p=0.05151; parametric statistical model, p=0.0122). Based on the body weight data MR1 appears to have less of an impact on the onset of disease in the G93A mouse model but has a dramatic effect in slowing down the rate of body weight loss from peak body weight until death.

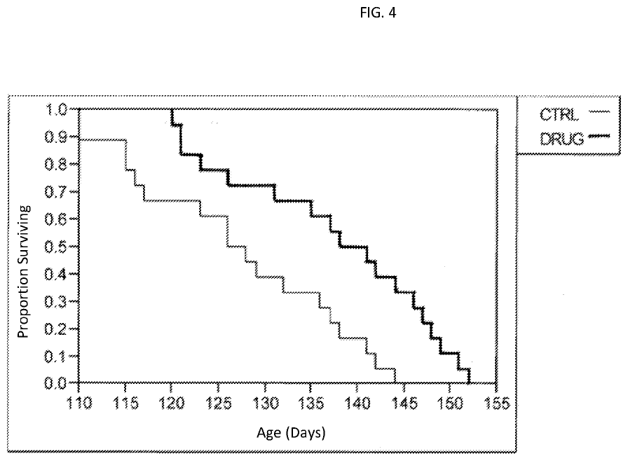

Treated animals survival time was later by 13 days than control animals. Comparative MR1-treated and control group time-to-event curves for the time from peak body weight to death are shown in FIG. 4. The median survival time for the control group was 128 days and the median survival for the MR1 treated group was 141 days. Control animals had a 2.8 to 3.2-fold greater risk of dying sooner than did MR1 treated animals. The delay is statistically significant when analyzed using several approaches. (Kaplan Meier, log rank (p=0.0040) and Wilcoxon test (p=0.0109); Cox proportional Hazard model (p=0.0060); parametric analysis, (p=0.0049)). 60 female and 36 male G93A mice were litter matched and randomly assigned to treatment or control groups. 30 of the female and 18 of the male mice were treated with MR1 starting at day 50. Study days are based on birth.

A single bolus injection of 5.22 mg/kg or 6.75 mg/kg of MR1 was administered intraperitoneally at day 50 in females or males, respectively. Subsequent to the bolus injection, females received weekly injections of 1 mg/kg of MR1, and males received weekly injections of 1.34 mg/kg of MR1 via IP injection. Doses were prepared in vehicle (phosphate buffered saline (PBS, pH 7.3)), to a total volume of 200 μl. Control animals where administered 200 μl PBS. Animals were monitored for body weight, and criteria for euthanization as previously described.

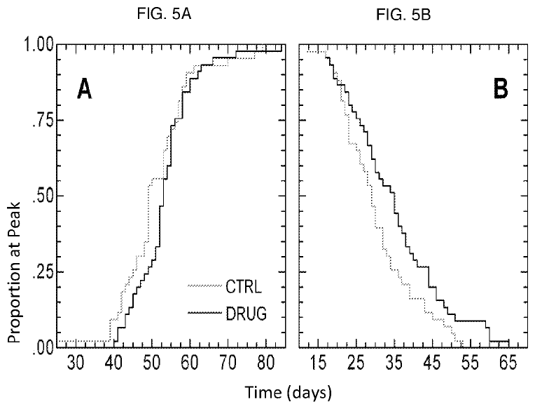

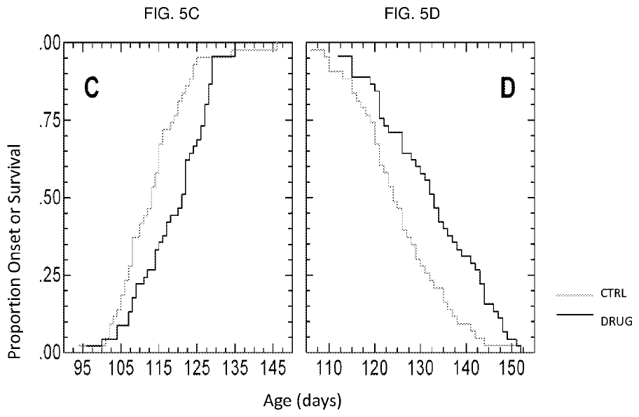

Comparative MR1-treated and control group time-to-event curves for the time from day 40 to the attainment of peak body weight are shown in FIG. 5A. The median time to peak body weight for the control group was 49 days compared to the MR1 treated group which was 53 days. This difference was not significant when analyzed by Kaplan Meier using the log rank and Wilcoxon statistical models, Cox proportional hazard, or parametric statistical tests.

Comparative MR1-treated and control group time-to-event curves for the time from peak body weight to death are shown in FIG. 5B. Time from peak body weight to death was statistically significantly later by 6 days in MR1 treated animals. The median time from peak body weight to death in the control group was 29 days whereas in the MR1 treated group it was 35 days. Significance for each of the analyses in this example was calculated in several ways in order to better evaluate significance. The delay is statistically significant when analyzed using several statistical models: Kaplan Meier, log rank (p=0.0413) and Wilcoxon, (p=0.0732); and the Cox proportional Hazard model (p=0.0460). Based on the body weight data, MR1 appears to have less of an impact on the onset of disease in the G93A mouse model but has a dramatic effect in slowing down the rate of body weight loss from peak body weight until death.

The time-to-event plots for the age at which MR1 and control group animals progressed to a neurological score of 2 and the number of days at a neurological score of 2 is shown in FIG. 5C. The time to attain a score of 2 in the control group was 113 days and for the MR1 treated group is 121 days. Based on the neurological score data, MR1 delays disease onset in the G93A mouse model by approximately 8 days and the delay is statistically significant when analyzed using several statistical models: Kaplan Meier, log rank (p=0.0038) and Wilcoxon; (p=0.0017); and the Cox proportional hazard model, (p=0.0010).

Treated animals survival time was later by 9 days than control animals. The median survival time for the control group was 124 days and the median survival for the MR1 treated group was 133 days, as shown in FIG. 5D. The delay is statistically significant when analyzed using several statistical models: Kaplan Meier, log rank (p=0.0043) and Wilcoxon test (p=0.0040); and the Cox proportional hazard model (p=0.0030).

It is understood that the examples and embodiments described herein are for illustrative purposes only and that various modifications or changes in light thereof will be suggested to persons skilled in the art and are to be included within the spirit and purview of this application and scope of the appended claims.

Nucleic acid sequences encoding the heavy chain and the light chain of the desired antibody were designed to be suitable for expression in mammalian cells such as Chinese Hamster Ovary (CHO) cells. The nucleic acids were then artificially synthesized and ligated into the antibody expression vector BPJPuro using standard molecular biology techniques. BPJPuro is a dual gene mammalian expression vector optimized for selectable and stable expression of immunoglobulins in Chinese Hamster Ovary (CHO) cells. The vector was then transfected into CHO cells and stable transfectants selected.

Similarly, a nucleic acid (SEQ ID NO: 10) encoding a heavy chain having the amino acid sequence of SEQ ID NO: 9, and a nucleic acid (SEQ ID NO: 8) encoding a light chain having the amino acid sequence of SEQ ID NO: 7, were synthesized and ligated into the antibody expression vector BPJPuro.

The resulting expression vector encoding the heavy and light chains was transfected into the CHO line (CHO SA, Cellectis SA, Paris, France) using liposome mediated transfection. Stable transfectants were isolated by puromycin selection and subcloned to provide clonal cell lines. Candidate cell lines were adapted to serum free suspension culture and screened for IgG production and robust growth. One of the cell lines was selected and named JB5, the cell line was cultured in a pilot scale bioreactor and the antibody JB5 was purified from conditioned medium by sequential concentration, Protein A/G affinity chromatography, and size exclusion chromatography.

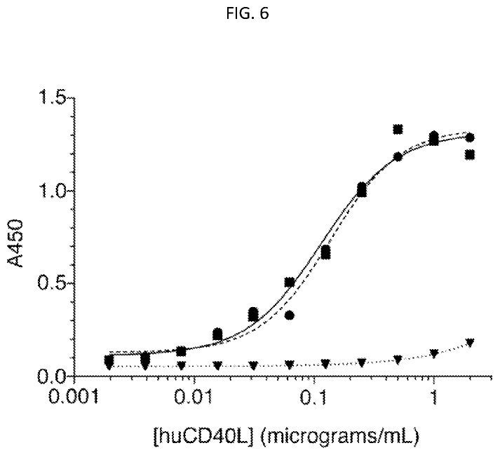

A three-part sandwich ELISA assay was used to determine binding kinetics of the JB5 antibody relative to the parental antibody hu5c8. All washes were performed using 3 washes of 250 μl of PBS. A 96-well polystyrene plate was coated with 100 μl/well of JB5 or hu5c8 antibody (2 μg/ml) for 16 hours at 4° C. The plate was washed and then blocked with 2% bovine serum albumin/PBS for 1 hour at room temperature. The plate was washed and recombinant human CD40L protein (Santa Cruz Biotechnology, Santa Cruz, California, USA) was added to the plate titrated out by 2-fold dilution starting at 2000 ng/ml. After binding and washing, the bound CD40L protein was detected using 100 μl a biotinylated goat anti-human CD40L polyclonal antibody (200 ng/ml) and 100 μl a streptavidin-horseradish peroxidase conjugate at 100 ng/ml. Colorimetric detection was performed with the chromagen TMB (3,3′,5,5′-tetramethylbenzidine) and spectrophotometric analysis of absorption at 450 nm. The resulting binding curves (FIG. 6) show that JB5 (circle) has highly similar CD40L binding relative to the parental antibody hu5c8 (square). The control protein CTLA4-IgG1 (triangle), having the same Fc domain as JB5 showed no significant binding. The calculated EC50 for hu5c8 and JB5 is 114 and 137 nM, respectively. JB5-R28K and JB5-K74R showed binding similar to that of JB5.

In order to make a comparison of CD40L binding of antibodies from all 16 clones versus 5c8 or AT-1501 (also known as tegoprubart, a humanized monoclonal antibody that inhibits CD40 ligand) a binding assay was run using 2 clones with 5c8 and AT-1501 run on the same 96-well assay plate. A three-part sandwich ELISA assay was used to determine the level of binding of the antibodies of the present disclosure in comparison to reference antibodies 5c8-19 and AT-1501. 96-well polystyrene plates were coated with recombinant human CD40L (BioLegend Cat. #591706) using 2 μg/ml in PBS and 50 μl/well was added to Costar 96-well ½ area high binding assay plates (Corning 3690) and incubated overnight at 4° C. Plates were blocked with (1×) PBS/1.0% BSA (140 μl/well) for 1 hour at room temperature to prevent background binding. Binding curves of 5C8 or AT1501 (from 2 μg/ml out serial 2-fold dilutions) were added (50 μl/well) and incubated for 1 hour at room temperature. Plates are washed and incubated with HRP-(Fab2) donkey anti-human IgG (Fc specific) (Jackson Immuno. 709-036-098) at a 1:10,000 dilution (50 μl/well) for 1 hour at room temperature. Plates were washed and TMB substrate (Surmodics BioFX TMBW-1000-01) was added (50 μl/well). Color development is stopped after 5 minutes at room temperature with 25 μl 2NH₂SO₄. Plates are read on Molecular Devices SpectraMax M5 plate reader using SoftMax Pro 6.2.2 program to determine absorbance at 450 nm.

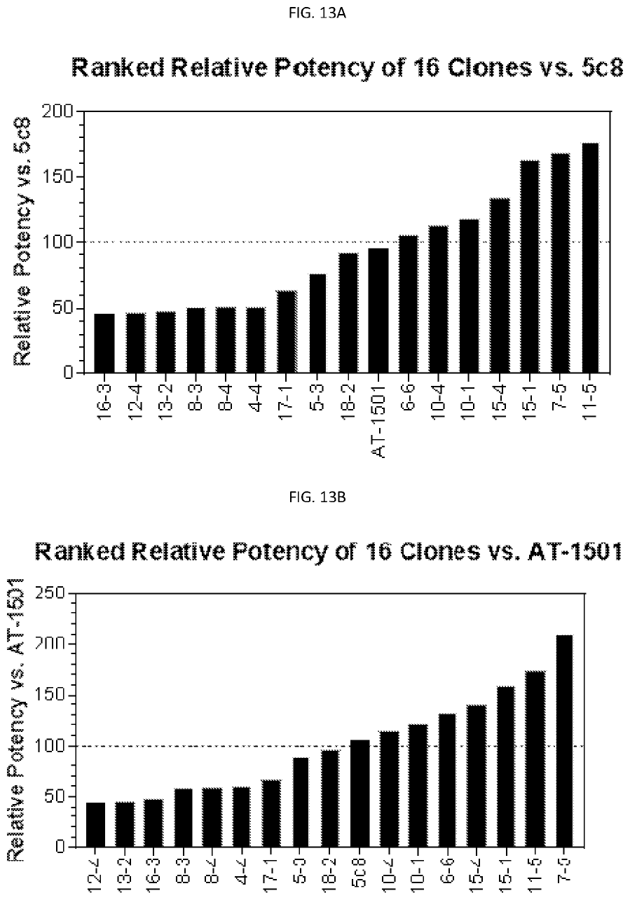

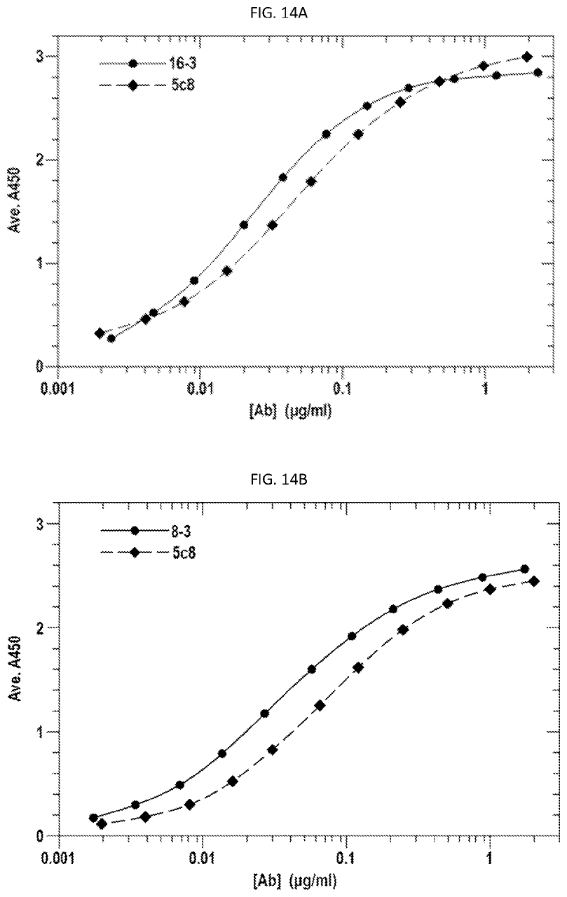

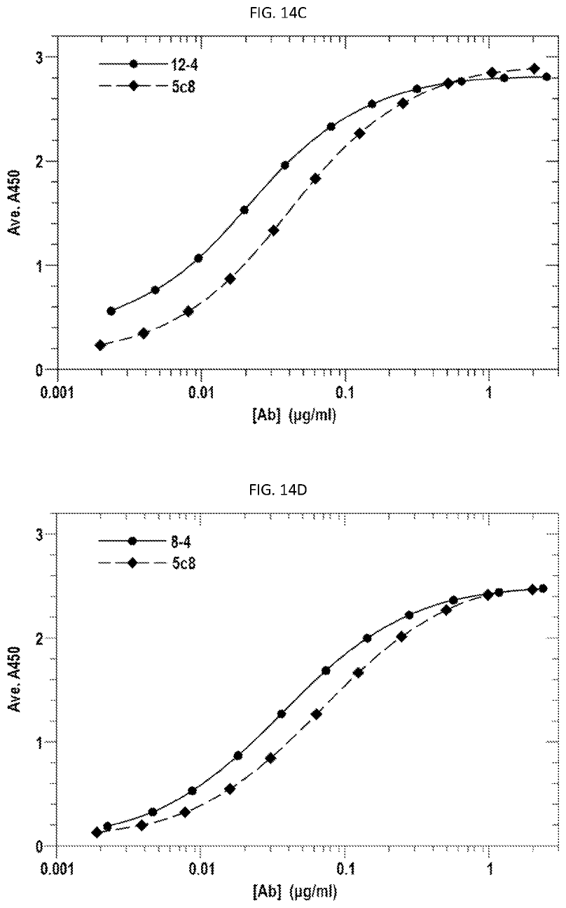

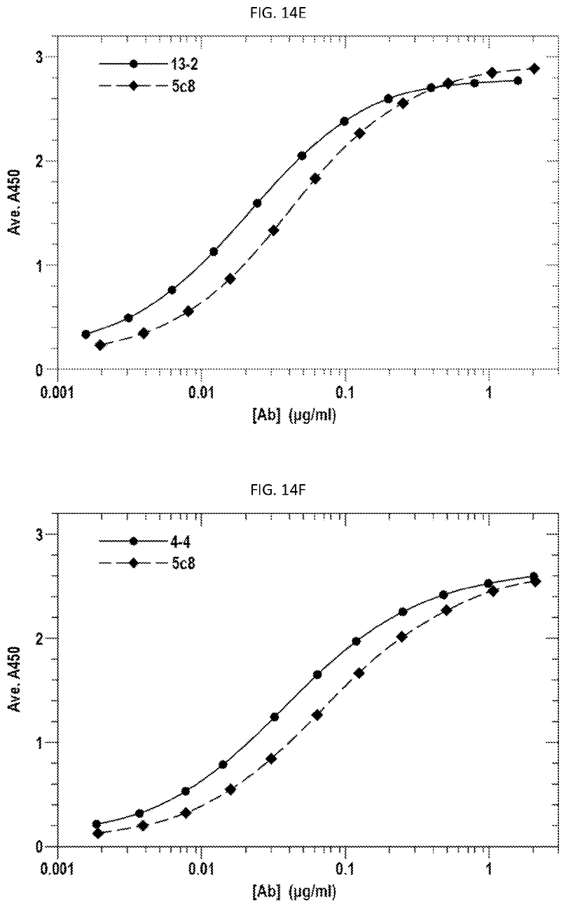

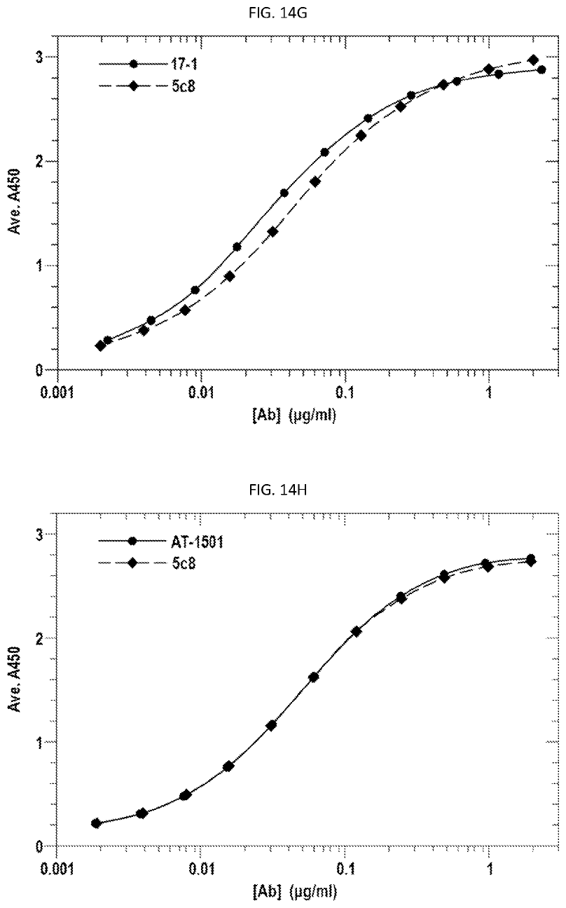

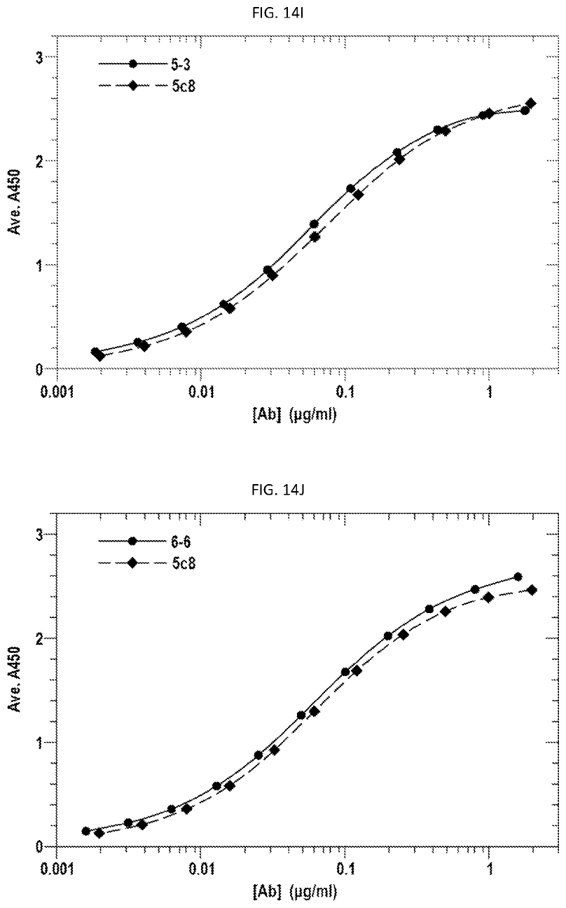

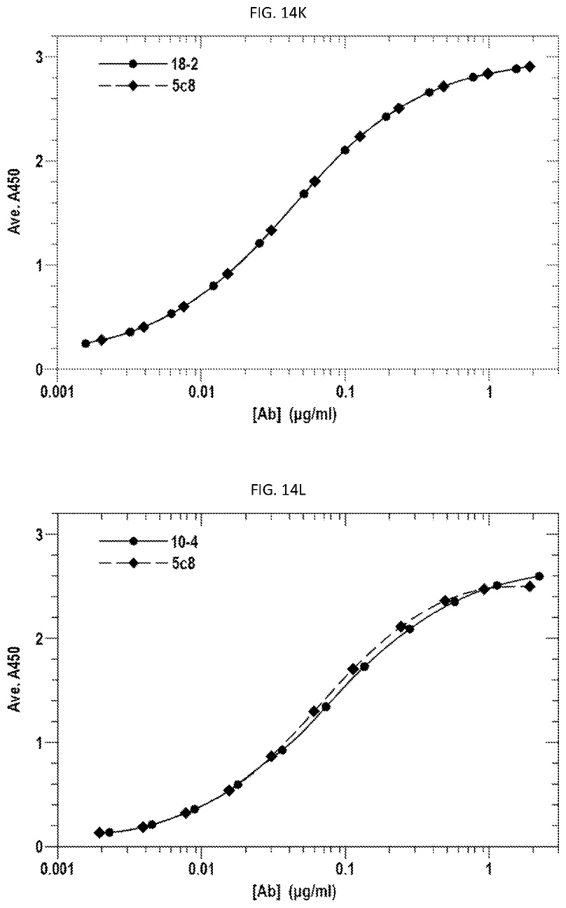

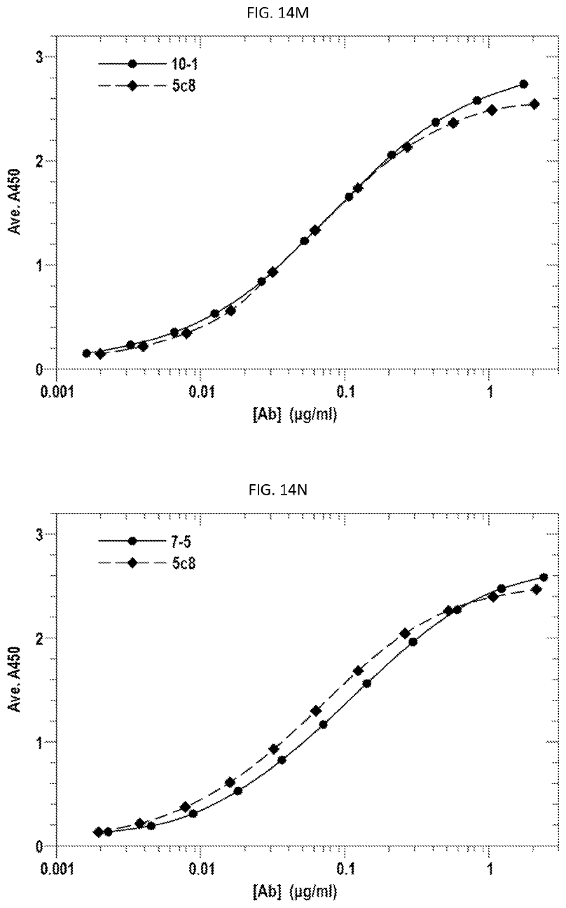

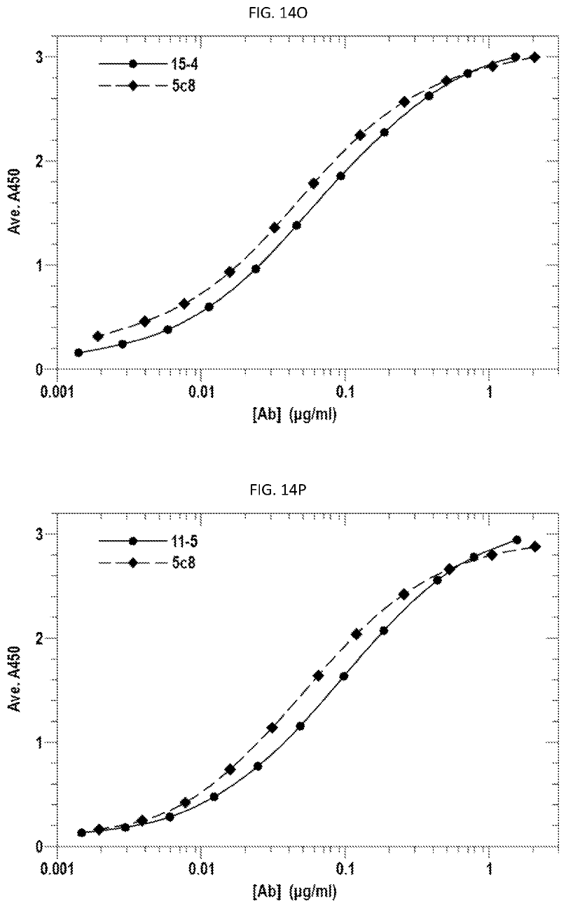

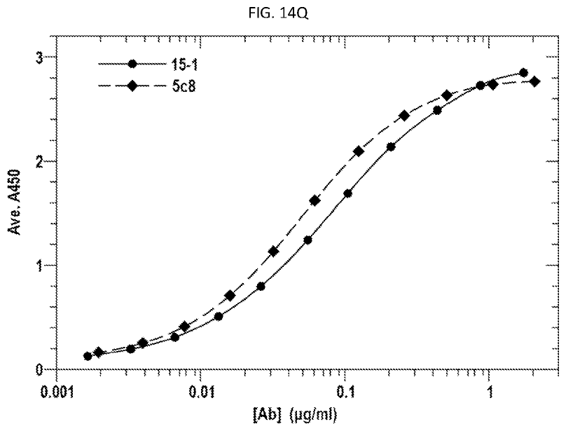

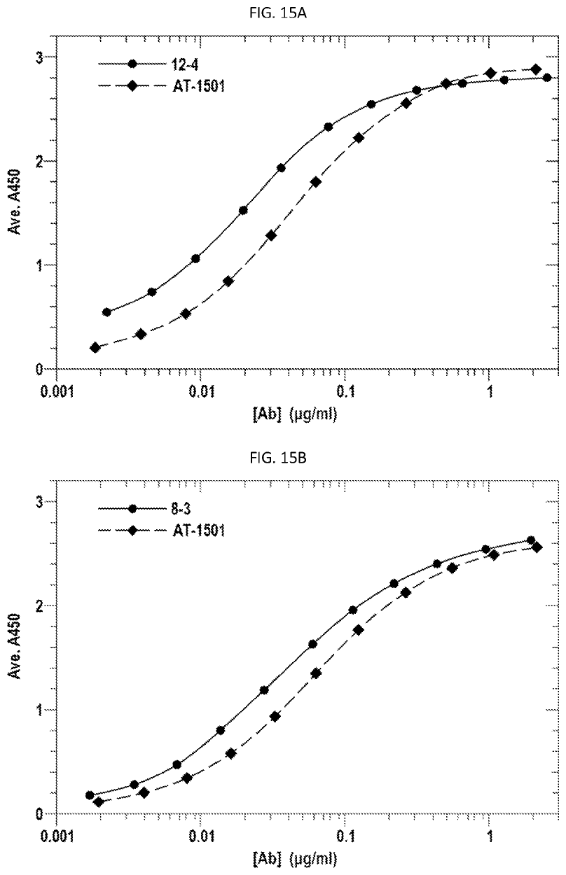

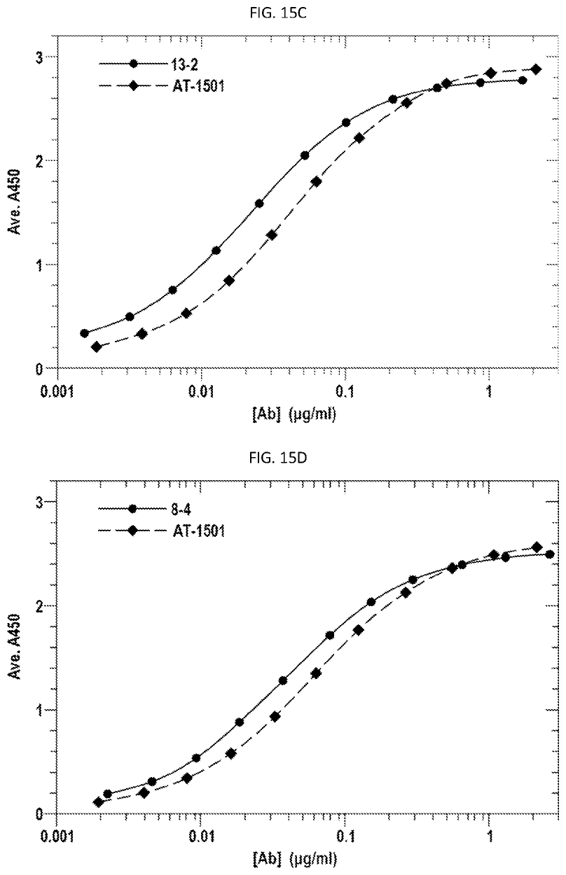

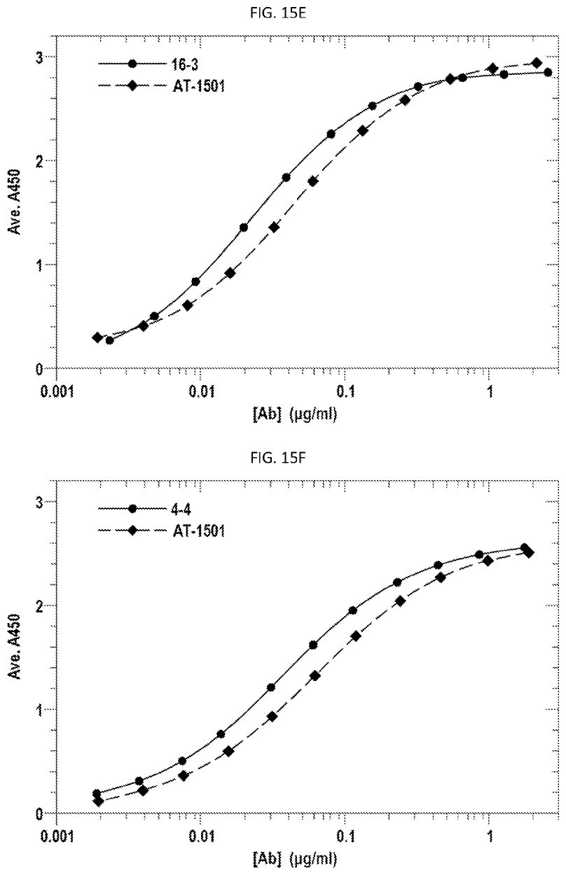

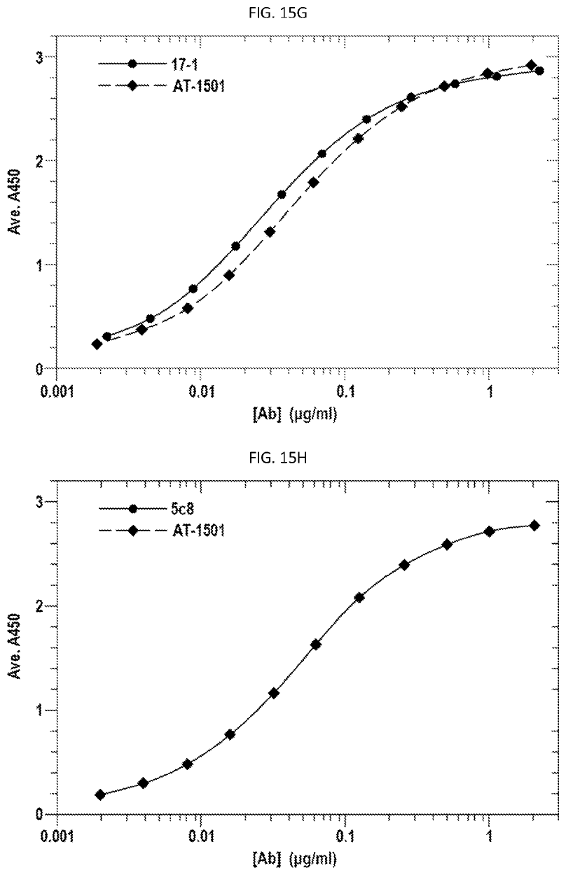

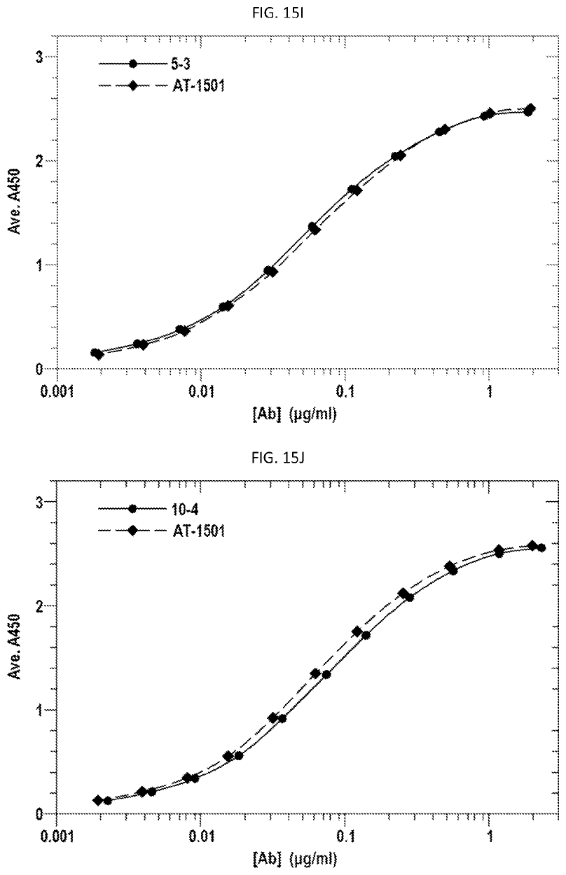

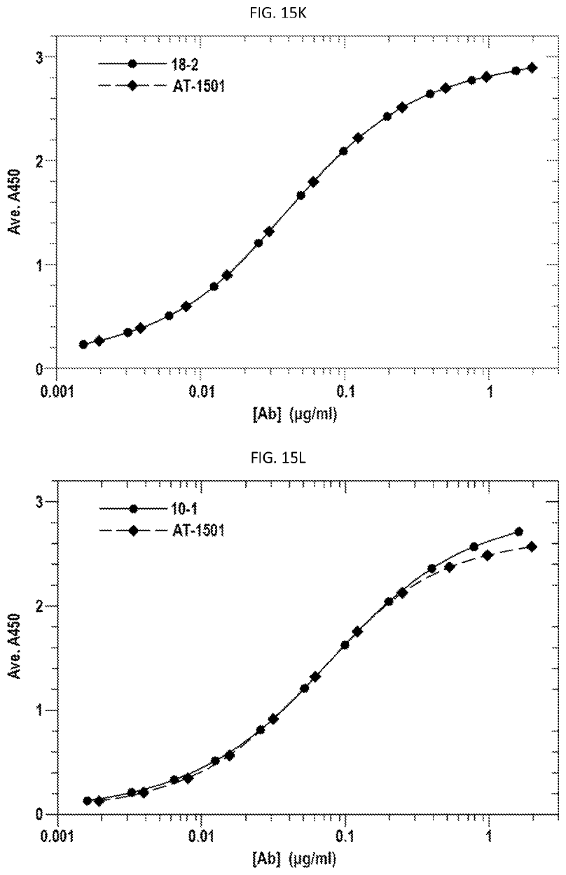

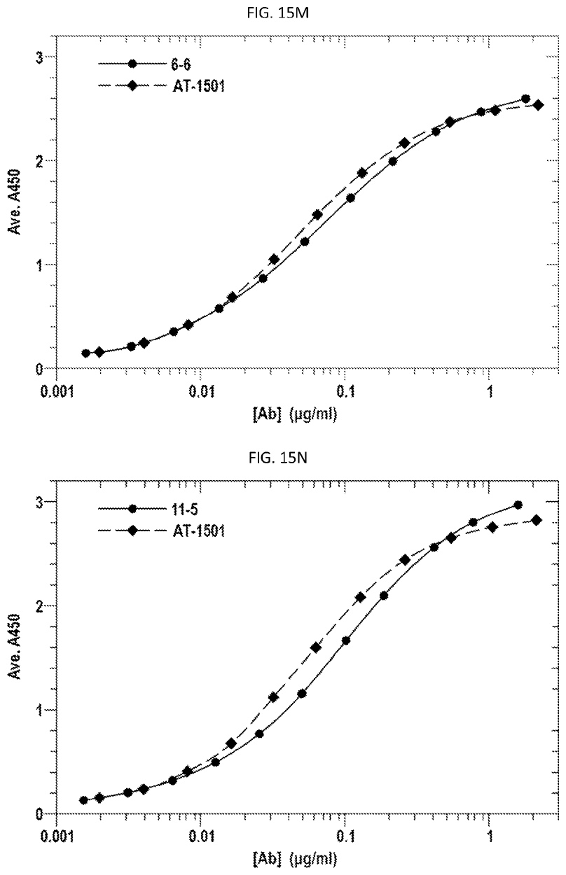

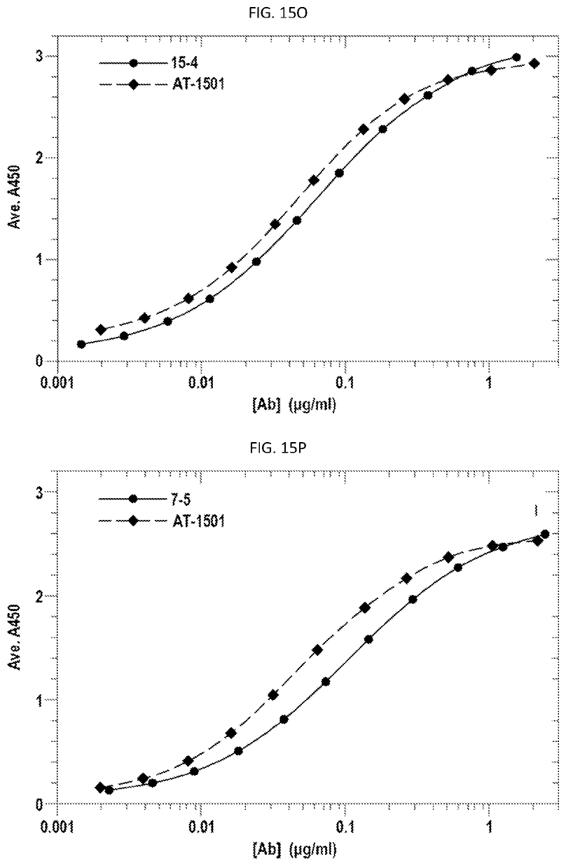

Relative CD40L binding potency was calculated as follows: [IC50 (clone)/IC50 (5c8 or AT-1501)×100%]. The ranked potency of the 16 clones versus 5c8 is shown in FIG. 13A and the ranked potency versus AT-1501 is shown in FIG. 13B. The resulting binding curves are shown in FIGS. 14A-14Q (comparison of 16 clones versus 5c8, FIG. 14H shows the comparison between AT-1501 and 5c8) and FIGS. 15A-15Q (comparison of the 16 clones versus AT-1501, FIG. 15H shows the comparison between AT-1501 and 5c8). The IC50, LCL, UCL and Relative Potency for each clone are shown in Table 1 (comparison with 5c8) and Table 2 (comparison with AT-1501).

TABLE 1

| FIG. No. | Clone | IC50 | LCL | UCL | Rel. Pot. |
|---|---|---|---|---|---|
| 14A | 16-3 | 0.022 | 0.017 | 0.026 | 46 |
| | 5c8 | 0.047 | 0.040 | 0.054 | |
| 14B | 8-3 | 0.036 | 0.028 | 0.043 | 51 |
| | 5c8 | 0.070 | 0.062 | 0.078 | |
| 14C | 12-4 | 0.020 | 0.013 | 0.027 | 47 |
| | 5c8 | 0.042 | 0.038 | 0.046 | |
| 14D | 8-4 | 0.036 | 0.030 | 0.041 | 51 |
| | 5c8 | 0.070 | 0.062 | 0.078 | |
| 14E | 13-2 | 0.020 | 0.013 | 0.027 | 48 |
| | 5c8 | 0.041 | 0.038 | 0.045 | |
| 14F | 4-4 | 0.037 | 0.029 | 0.045 | 51 |
| | 5c8 | 0.072 | 0.064 | 0.081 | |
| 14G | 17-1 | 0.028 | 0.023 | 0.033 | 63 |
| | 5c8 | 0.044 | 0.036 | 0.053 | |
| 14H | AT-1501 | 0.049 | 0.043 | 0.056 | 95 |
| | 5c8 | 0.052 | 0.046 | 0.058 | |
| 14I | 5-3 | 0.055 | 0.047 | 0.063 | 76 |
| | 5c8 | 0.072 | 0.064 | 0.081 | |
| 14J | 6-6 | 0.065 | 0.059 | 0.070 | 106 |
| | 5c8 | 0.061 | 0.054 | 0.068 | |
| 14K | 18-2 | 0.041 | 0.036 | 0.046 | 92 |
| | 5c8 | 0.044 | 0.036 | 0.053 | |
| 14L | 10-4 | 0.075 | 0.065 | 0.086 | 113 |
| | 5c8 | 0.067 | 0.058 | 0.076 | |
| 14M | 10-1 | 0.079 | 0.070 | 0.089 | 119 |
| | 5c8 | 0.067 | 0.058 | 0.076 | |
| 14N | 7-5 | 0.102 | 0.094 | 0.111 | 168 |
| | 5c8 | 0.061 | 0.054 | 0.068 | |
| 14O | 15.4 | 0.063 | 0.056 | 0.069 | 133 |
| | 5c8 | 0.047 | 0.040 | 0.054 | |
| 14P | 11-5 | 0.092 | 0.071 | 0.112 | 177 |
| | 5c8 | 0.052 | 0.046 | 0.058 | |
| 14Q | 15-1 | 0.081 | 0.073 | 0.089 | 163 |
| | 5c8 | 0.050 | 0.046 | 0.053 | |

TABLE 2

| FIG. No. | Clone | IC50 | LCL | UCL | Rel. Pot. |
|---|---|---|---|---|---|
| 15A | 12-4 | 0.020 | 0.013 | 0.027 | 45 |
| | AT-1501 | 0.044 | 0.039 | 0.049 | |
| 15B | 8-3 | 0.036 | 0.028 | 0.043 | 59 |
| | AT-1501 | 0.060 | 0.053 | 0.068 | |
| 15C | 13-2 | 0.020 | 0.013 | 0.027 | 45 |
| | AT-1501 | 0.044 | 0.038 | 0.049 | |
| 15D | 8-4 | 0.036 | 0.030 | 0.041 | 59 |
| | AT-1501 | 0.060 | 0.053 | 0.068 | |
| 15E | 16-3 | 0.022 | 0.017 | 0.026 | 48 |
| | AT-1501 | 0.045 | 0.039 | 0.051 | |
| 15F | 4-4 | 0.037 | 0.029 | 0.045 | 60 |
| | AT-1501 | 0.062 | 0.054 | 0.070 | |
| 15G | 17-1 | 0.028 | 0.023 | 0.033 | 66 |
| | AT-1501 | 0.042 | 0.035 | 0.050 | |
| 15H | 5c8 | 0.052 | 0.046 | 0.058 | 105 |
| | AT-1501 | 0.049 | 0.043 | 0.056 | |
| 15I | 5-3 | 0.055 | 0.047 | 0.063 | 89 |
| | AT-1501 | 0.062 | 0.054 | 0.070 | |
| 15J | 10-4 | 0.075 | 0.065 | 0.086 | 115 |
| | AT-1501 | 0.066 | 0.056 | 0.076 | |
| 15K | 18-2 | 0.041 | 0.036 | 0.046 | 96 |
| | AT-1501 | 0.042 | 0.035 | 0.050 | |
| 15L | 10-1 | 0.079 | 0.070 | 0.089 | 121 |
| | AT-1501 | 0.066 | 0.056 | 0.076 | |
| 15M | 6-6 | 0.065 | 0.059 | 0.070 | 132 |
| | AT-1501 | 0.049 | 0.047 | 0.051 | |
| 15N | 11-5 | 0.092 | 0.071 | 0.112 | 174 |
| | AT-1501 | 0.053 | 0.050 | 0.056 | |
| 15O | 15-4 | 0.063 | 0.056 | 0.069 | 139 |
| | AT-1501 | 0.045 | 0.039 | 0.051 | |
| 15P | 7-5 | 0.102 | 0.094 | 0.111 | 210 |
| | AT-1501 | 0.049 | 0.047 | 0.051 | |
| 15Q | 15-1 | 0.081 | 0.073 | 0.089 | 159 |
| | AT-1501 | 0.051 | 0.046 | 0.055 | |

The 16 VH/VL antibody clones constructed with an IgG1 Fc having two mutations P238S and N297G (SEQ ID NO: 21). These antibody clones were assayed for Fc effector function for binding to human FcγRI, FcγRIIa and FCγRIIIa.

Anti-CD40L antibodies (Abatacept included as negative control) are diluted to 2 g/ml in (1×) PBS and 50 μl/well was added to Costar 96-well ½ area high binding assay plates (Corning 3690) for overnight incubation at 4° C. Plates were blocked with (1×) PBS/1.0% BSA (140 μl/well) for 1 hour at room temperature to prevent background binding. Binding curves of recombinant human FcγRI, IIa, IIIa and IIIb (from 5 μg/ml out serial 2-fold dilutions) were added (50 μl/well) and incubated for 1 hour at room temperature. Plates were washed and incubated with mouse anti-human CD16 (anti-FcRIII); CD32 (anti-FcRIIa) or CD64 (anti-FcRI) (eBioSciences/Invitrogen 14-0168-82; 16-0329-81; 14-0649-82) at 2 μg/ml (50 μl/well) for 1 hour at room temperature. Plates were washed and incubated with HRP-(Fab2) goat anti-mouse IgG (Fc specific) (Jackson Immuno. 116-036-071) at a 1:10,000 dilution (50 μl/well) for 1 hour at room temperature. Plates were washed and TMB substrate (Surmodics BioFX TMBW-1000-01) was added (50 μl/well). Color development was stopped after 5 mins at room temperature with (25 μl/well) 2NH₂SO₄. Plates were read on Molecular Devices SpectraMax M5 plate reader using SoftMax Pro 6.2.2 program to determine absorbance at 450 nm.

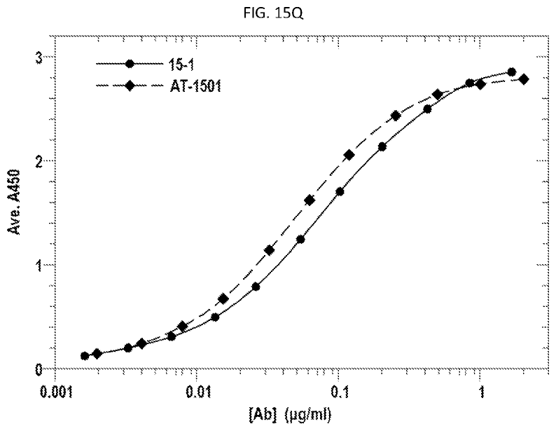

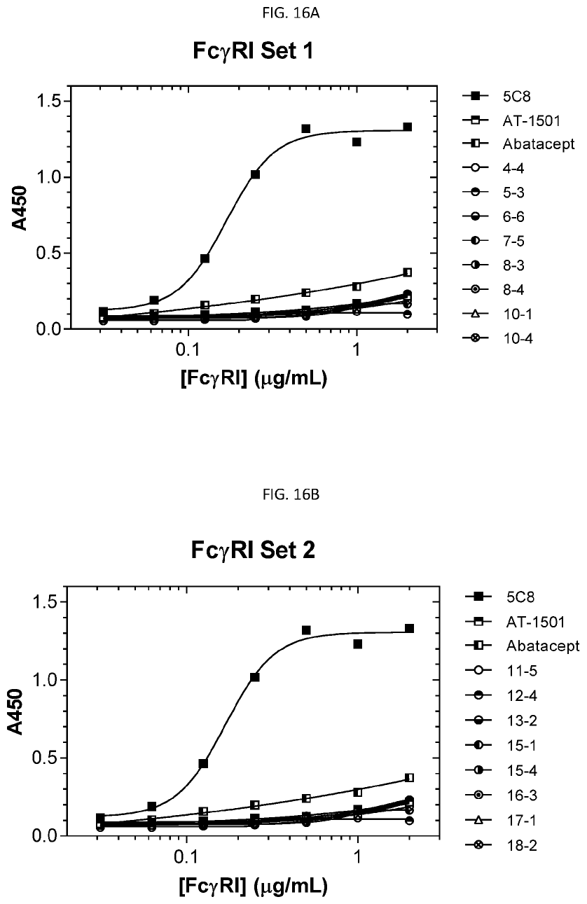

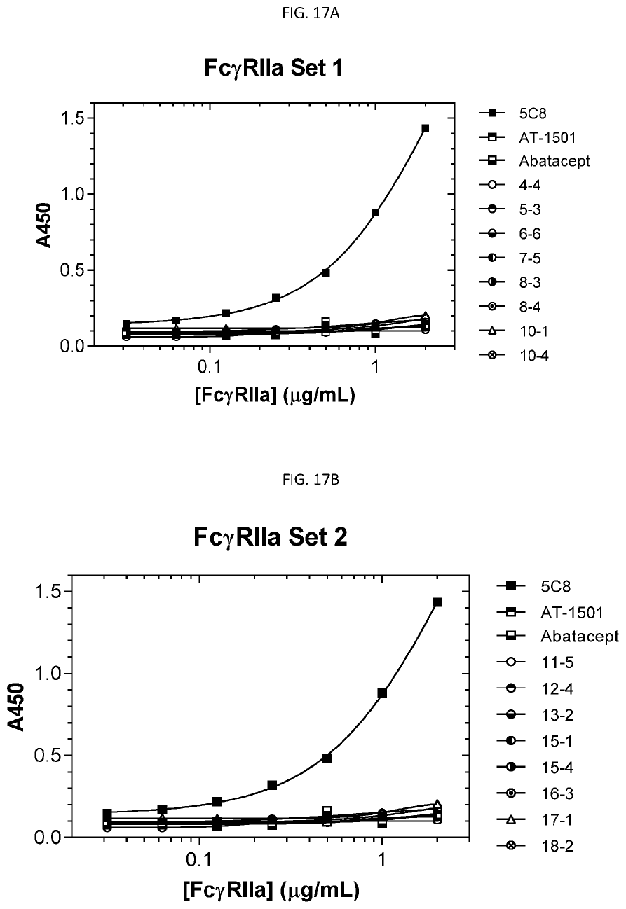

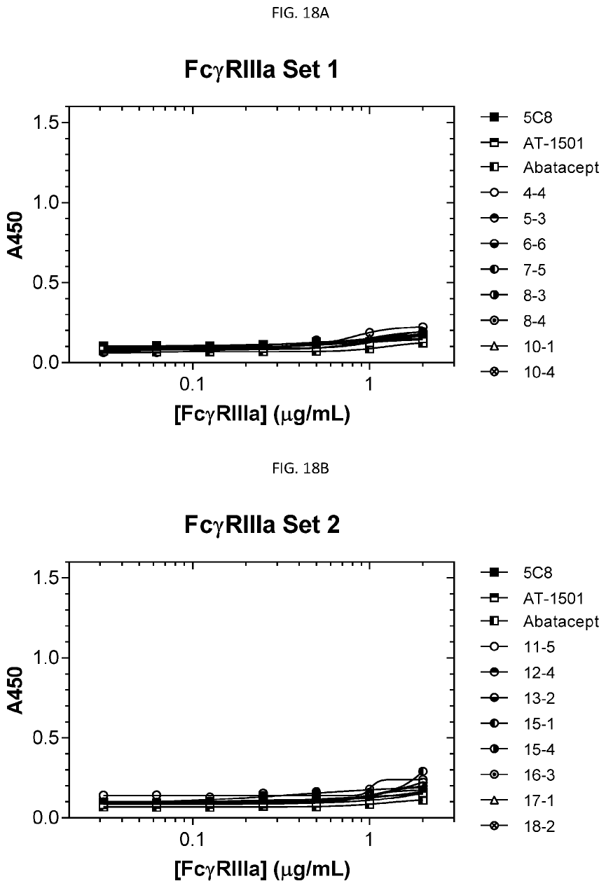

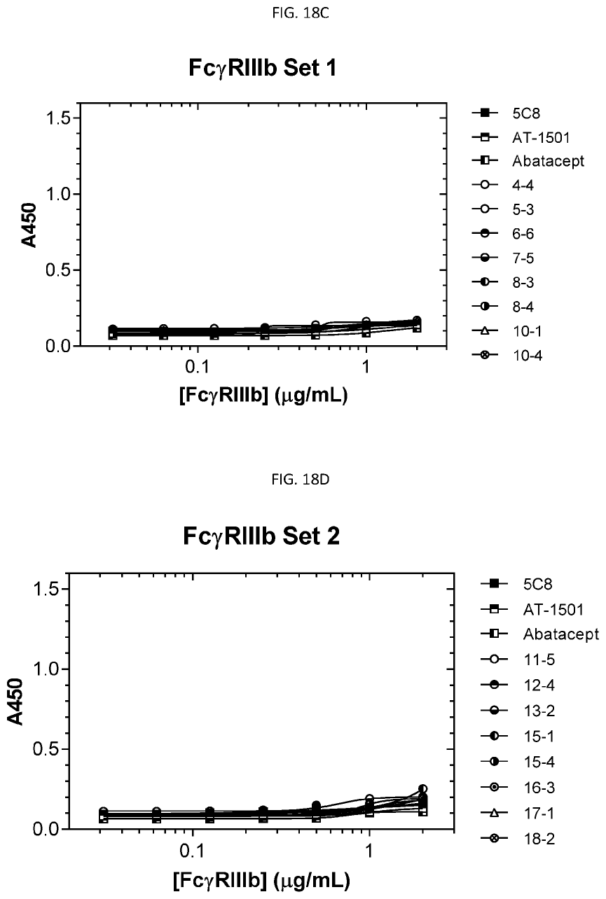

As can be seen in the figures, the clones disclosed in this application were negative for binding to the three Fc receptors while 5c8 bound to FcγRI, FcγRIIa but not to FcγRIIIa or FcγRIIIb. Each of FIGS. 16A and 16B show the binding to FcγRIa for eight antibody clones compared with the binding curves of 5c8, AT-1501 and Abatacept. Each of FIGS. 17A and 17B show the binding to FcγRIIa for eight antibody clones compared with the binding curve of 5c8, AT-1501 and Abatacept. Each of FIGS. 18A and 18B show the binding to FcγRIIIa for eight antibody clones compared with the binding curves of 5c8, AT-1501 and Abatacept. Each of FIGS. 18C and 18D show the binding to FcγRIIIb for eight antibody clones compared with the binding curves of 5c8, AT-1501 and Abatacept. (FIGS. 16A, 17A, 18A and 18B show results from antibody clones 4-4, 5-3, 6-6, 7-5, 8-3, 8-4, 10-1 and 10-4; FIGS. 16B, 17B, 18C and 18D show results from antibody clones 11-5, 12-4, 13-2, 15-1, 15-4, 16-3, 17-1 and 18-2).

Anti-CD40L antibodies (Abatacept included as negative control) were diluted to 2 μg/ml in (1×) PBS and 50 μl/well was added to Costar 96-well ½ area high binding assay plates (Corning 3690) for overnight incubation at 4° C. Plates were blocked with (1×) PBS/1.0% BSA (140 μl/well) for 1 hour at room temperature to prevent background binding. Binding curves of natural human C1q protein (Abcam ab96363) from 10 μg/ml out serial 2-fold dilutions were added (50 μl/well) for 1 hour at room temperature. Plates were washed and HRP-sheep anti-human C1q (Abcam ab46191) is added at a 1:400 dilution (50 μl/well) and incubated for 1 hour at room temperature. Plates were washed and TMB substrate (Surmodics BioFX TMBW-1000-01) is added (50 μl/well). Color development was stopped after 5 minutes at room temperature with (25 μl/well) 2NH₂SO₄. Plates were read on Molecular Devices SpectraMax M5 plate reader using SoftMax Pro 6.2.2 program to determine absorbance at 450 nm.

As can be seen in the figures, the antibody from all sixteen antibody clones disclosed in this application were negative for binding to C1q while 5c8 showed significant binding. Each of FIGS. 19A and 19B show the binding to C1q for eight antibody clones compared with the binding curves of 5c8, AT-1501 and Abatacept. FIG. 19A shows results from antibody clones 4-4, 5-3, 6-6, 7-5, 8-3, 8-4, 10-1 and 10-4; FIG. 19B shows results from antibody clones 11-5, 12-4, 13-2, 15-1, 15-4, 16-3, 17-1 and 18-2.

Because the genetic expression data indicated the involvement of the co-stimulator pathway, the efficacy of MR1 was evaluated in the G93A model. MR1 binds to CD40L thus blocking its interaction of CD40 which participates in the co-stimulatory pathway involved in an immune response. It has been reported in the literature that MR1 is efficacious for the treatment of rheumatoid arthritis and graft versus host disease both of which have a strong immunologic component. Rheumatoid arthritis is an autoimmune disease and graft versus host disease arises when the host's body mounts a vigorous immune response against the graft tissue.

Methods according to the present disclosure include methods of treating a patient with an autoimmune disorder, including a kidney related autoimmune disorder, such as a kidney-related disease, disorder, or complication by administering to the patient a compound that blocks the interaction of CD40L and CD40 and/or blocks the interaction of CD28 and CD80 and/or blocks the interaction of CD28 and CD86. In some embodiments, the methods include treating a patient having colitis, drug induced lupus nephritis, lupus nephritis, nephropathy, chronic kidney disease, graft versus host disease, renal transplantation, immune graft response, transplant rejection, atherosclerosis, diabetic kidney disease, acute kidney injury, kidney failure, polycystic kidney disease, hematuria, Fabry disease, lupus kidney disease, Alport's syndrome, IgM nephropathy, interstitial nephritis, Membranoproliferative Glomerulonephritis, simple kidney cysts, kidney infections, kidney stones, or IgA nephropathy, by administering to the patient a compound that blocks the interaction of CD40L and CD40. In some embodiments, the methods include treating a patient by administering an anti-CD40L antibody. In some embodiments, the methods include treating a patient having an autoimmune disease. Non-limiting examples of autoimmune diseases include systemic lupus erythematous, type-1 diabetes, myasthenia gravis, inflammatory bowel disease, immune thrombocytopenia purpura, rheumatoid arthritis, psoriasis, Addison's disease, Crohn's disease, uveitis, multiple sclerosis, hemolytic anemia, and Graves' disease.

Therapeutic compounds provided herein include any compound that blocks the interaction of CD40 with CD40L. For example, a number of animal studies describe agents capable of interrupting CD40:CD40L binding (see for example US2005158314 and U.S. Pat. No. 7,173,046 which are hereby incorporated by reference). Numerous anti-CD40L antibodies have been produced and characterized (see, e.g., U.S. Pat. No. 5,876,950) to Bristol-Myers Squibb, which is hereby incorporated by reference), Anti-CD40L antibodies useful in the methods of the present disclosure include, but are not limited to, MR1, a hamster monoclonal antibody available from Taconic (Hudson, NY) and BD Biosciences (San Jose, CA); 5c8, a humanized antibody described in U.S. Pat. No. 5,474,771 (which is hereby incorporated by reference); a hamster human chimeric antibody, IDEC 131/E6040 is a humanized monoclonal antibody comprising human gamma-1 heavy chains and human kappa-light chains with CDRs of murine monoclonal antibody clone 24-31, commercially available from Ancell (catalog X 353-020, Bayport, Minn.); ABI 793; Sgn-40; ImxM90 (Immunex); ImxM91 (Immunex); ImxM92 (Immunex); and an anti-CD40L mAb commercially available from Genzyme (Cambridge, Mass., catalog No. 80-3703-01). Also commercially available is an anti-CD40L mAb from PharMingen (San Diego, Catalog #33580D). Embodiments according to the disclosure include methods of treating a patient with a kidney-related autoimmune disease, disorder, or complication, comprising administering a therapeutically effective amount of any compound capable of binding to CD40L, such as an anti-CD40L antibody. In some embodiments, the methods include treating a patient with a kidney-related autoimmune disease, disorder, or complication, comprising administering a therapeutically effective amount of an anti-CD40L antibody selected from MR1, 5c8, IDEC 131/E6040, clone 24-31, ABI 793, ImxM90, ImxM91, ImxM92, or Sgn-40. In some embodiments, the antibody is 5c8. In some embodiments, the antibody is MR1.

In some embodiments the kidney-related disease, disorder, or complication is colitis, drug induced lupus nephritis, lupus nephritis, nephropathy, chronic kidney disease, graft versus host disease, renal transplantation, immune graft response, transplant rejection, atherosclerosis, diabetic kidney disease, acute kidney injury, kidney failure, polycystic kidney disease, hematuria, Fabry disease, lupus kidney disease, Alport's syndrome, IgM nephropathy, interstitial nephritis, Membranoproliferative Glomerulonephritis, simple kidney cysts, kidney infections, kidney stones, or IgA nephropathy, by administering to the patient a compound that blocks the interaction of CD40L and CD40. In some embodiments, the methods of treating a patient is by administering an anti-CD40L antibody. In some embodiments, the methods include treating a patient having an autoimmune disease. Non-limiting examples of autoimmune diseases include systemic lupus erythematous, type-1 diabetes, myasthenia gravis, inflammatory bowel disease, immune thrombocytopenia purpura, rheumatoid arthritis, psoriasis, Addison's disease, Crohn's disease, uveitis, multiple sclerosis, hemolytic anemia, and Graves' disease. In some embodiments, the anti-CD40L antibody is MR1. In some embodiments, the anti-CD40L antibody is 5c8.

In some embodiments, the methods of treatment comprises administering a therapeutically effective amount of an anti-CD40 antibody. In some embodiments the anti-CD40L compounds are Fab fragments, F(ab′) 2, F(ab′), single chain antibodies, polypeptides, fusion constructs of polypeptides and the like. In some embodiments the compounds are small molecule compounds that are capable of blocking the CD40:CD40L interaction. In some embodiments these compounds include BIO3417, or any of the compounds disclosed in U.S. Pat. No. 7,173,046, having the ability to block the CD40:CD40L interaction.

The compounds that block the CD40:CD40L interaction may be administered in combination with other compounds. Thus, in some embodiments, the methods of treating a patient as described herein include administering a compound that blocks the interaction of CD40L and CD40 in combination with a compound that blocks the interaction between CD80 and CD28. In some embodiments, the methods include administering a compound that blocks the interaction of CD40L and CD40 in combination with a compound that blocks the interaction between CD86 and CD28. In some embodiments, the compound that blocks the interaction of CD80 and CD28 is galiximab, or H1f1 & h3d1, or 16C10, or 7C10. In some embodiments, the compound that blocks the interaction between CD86 and CD28 is a CTLA4-Ig protein conjugate, such as 34batacept or belatacept. Embodiments according to the present disclosure also include methods of treating a patient with an autoimmune disorder, such as a kidney associated autoimmune disorder, including kidney-related disease, disorder, or complication comprising administering a compound that blocks the interaction of CD40L and CD40 in combination with a compound that blocks the interaction between CD80 and CD28 or administering a therapeutically effective amount of a compound that blocks the interaction of CD40L and CD40 in combination with a compound that blocks the interaction between CD86 and CD28, wherein the kidney-related disease, disorder or complication is colitis, drug induced lupus nephritis, lupus nephritis, nephropathy, chronic kidney disease, graft versus host disease, renal transplantation, immune graft response, transplant rejection, atherosclerosis, diabetic kidney disease, acute kidney injury, kidney failure, polycystic kidney disease, hematuria, Fabry disease, lupus kidney disease, Alport's syndrome, IgM nephropathy, interstitial nephritis, Membranoproliferative Glomerulonephritis, simple kidney cysts, kidney infections, kidney stones, or IgA nephropathy, by administering to the patient a compound that blocks the interaction of CD40L and CD40. In some embodiments, the methods include administering an anti-CD40L antibody. In some embodiments, the methods include treating a patient having an autoimmune disease. Non-limiting examples of autoimmune diseases include systemic lupus erythematous, type-1 diabetes, myasthenia gravis, inflammatory bowel disease, immune thrombocytopenia purpura, rheumatoid arthritis, psoriasis, Addison's disease, Crohn's disease, uveitis, multiple sclerosis, hemolytic anemia, and Graves' disease. In some embodiments, the compound that blocks the interaction of CD40L and CD40 is MR1 and the compound that blocks the interaction between CD28 and CD86 or between CD28 and CD80 is abatacept, galiximab or belatacept. In some embodiments, the compound that blocks the interaction of CD40L and CD40 is 5c8. In some embodiments, the methods include administering a therapeutically effective amount of MR1 in combination with abatacept or belatacept. In some embodiments, the methods include administering a therapeutically effective amount of 5c8 in combination with abatacept or belatacept.

### Kidney-Related Autoimmune Diseases, Disorders, and Conditions

The present disclosure relates to treatments of subjects with one or more autoimmune disease, including a kidney-related autoimmune disease, disorder, and/or condition. In some embodiments, the autoimmune disease is a kidney-related disease, disorder, or condition. In some embodiments, the kidney-related disease, disorder, or condition is a complication following a kidney transplant. In some embodiments, the kidney-related disease, disorder, or condition is kidney transplant rejection. In some embodiments, the kidney-related disease, disorder, or condition is graft-vs-host disease. In some embodiments, the kidney-related disease, disorder, or condition is a chronic kidney disease. In some embodiments, the kidney-related disease, disorder, or condition is a nephropathy. In some embodiments, the kidney-related disease, disorder, or condition is IgA nephropathy. In some embodiments, the kidney-related disease, disorder, or condition is an autoimmune disease of the kidney. In some embodiments, the kidney-related disease, disorder, or condition is inflammation or involves inflammation.

It will be understood that the compounds disclosed herein targeting CD40 or CD40L can be used against any disease, disorder, or condition in which the kidney is impaired. Non-limiting examples of kidney-related diseases, disorders, and conditions include: colitis, drug induced lupus nephritis, lupus nephritis, nephropathy, chronic kidney disease, graft versus host disease, renal transplantation, immune graft response, transplant rejection, atherosclerosis, diabetic kidney disease, acute kidney injury, kidney failure, polycystic kidney disease, hematuria, Fabry disease, lupus kidney disease, Alport's syndrome, IgM nephropathy, interstitial nephritis, Membranoproliferative Glomerulonephritis, simple kidney cysts, kidney infections, kidney stones, IgA nephropathy, systemic lupus erythematous, type-1 diabetes, myasthenia gravis, inflammatory bowel disease, immune thrombocytopenia purpura, rheumatoid arthritis, psoriasis, Addison's disease, Crohn's disease, uveitis, multiple sclerosis, hemolytic anemia, Graves' disease, Abderhalden-Kaufmann-Lignac syndrome (Nephropathic Cystinosis), Abdominal Compartment Syndrome, Acetaminophen-induced Nephrotoxicity, Acute Kidney Failure/Acute Kidney Injury, Acute Injury with Loin Pain after Anaerobic Exercise (ALPE), Renal Hypouricemia, Acute Lobar Nephronia, Acute Phosphate Nephropathy, Acute Tubular Necrosis, Adenine Phosphoribosyltransferase Deficiency, Adenovirus Nephritis, Alagille Syndrome, Alkaptonuria, Alport Syndrome, Alstrom Syndrome, Amyloidosis, ANCA Vasculitis Related to Endocarditis and Other Infections, Angiomyolipoma, Anabolic Steroid Abuse, Analgesic Nephropathy, Anorexia Nervosa and Kidney Disease, Angiotensin Antibodies and Focal Segmental Glomerulosclerosis, Anticoagulant-related Nephropathy, Antiphospholipid Syndrome, Anti-LRP2 Nephropathy, Anti-Brush Border Nephropathy, Anti-TNF-α Therapy-related Glomerulonephritis, APOLI Mutations, Apparent Mineralocorticoid Excess Syndrome, Aristolochic Acid Nephropathy, Chinese Herbal Nephropathy, Balkan Endemic Nephropathy, Arteriovenous Malformations and Fistulas of the Urologic Tract, Atypical Hemolytic Uremic Syndrome, Autosomal Dominant Hypocalcemia, Bardet-Biedl Syndrome, Bartter Syndrome, Bath Salts and Acute Kidney Injury, Beer Potomania, Beeturia, β-Thalassemia Renal Disease, Bile Cast Nephropathy, Birt-Hogg-Dubé Syndrome, BK Polyoma Virus Nephropathy in the Native Kidney, Bladder Rupture, Bladder Sphincter Dyssynergia, Bladder Tamponade, Bodybuilder's Glomerulopathy, Border-Crossers' Nephropathy, Bourbon Virus and Acute Kidney Injury, BRASH Syndrome, Acute Renal Dysfunction, Byetta and Renal Failure, C1q Nephropathy, C3 Glomerulopathy, C4 Glomerulopathy, CAKUT (Congenital Anomalies of the Kidney and Urologic Tract), Calyceal Diverticulum, Calcineurin Inhibitor Nephrotoxicity, Callilepsis Laureola Poisoning, Cannabinoid Hyperemesis Acute Renal Failure, Capillary Leak Syndrome, Carconoid Syndrome and Renal Failure, Cardiorenal syndrome, Carfilzomib-Induced Renal Injury, CFHR5 nephropathy, Charcot-Marie-Tooth Disease with Glomerulopathy, Cholesterol Emboli, Churg-Strauss syndrome, Chyluria, Ciliopathy, Cisplatin Nephrotoxicity, Cold Diuresis, Colistin Nephrotoxicity, Collagenofibrotic Glomerulopathy, Collapsing Glomerulopathy, Collapsing Glomerulopathy Related to CMV, Combination Antiretroviral (cART) Related-Nephropathy, Congenital Adrenal Hyperplasia, 17-Alpha-Hydroxylase/7 20-Lyase Deficiency, Congenital Anomalies of the Kidney and Urinary Tract (CAKUT), Congenital Nephrotic Syndrome, Congestive Renal Failure, Conorenal syndrome (Mainzer-Saldino Syndrome or Saldino-Mainzer Disease), Contrast Nephropathy, Copper Sulphate Intoxication, Coronavirus (COVID-19) Associated Kidney Failure and Kidney Disease, Cortical Necrosis, Crizotinib-related Acute Kidney Injury, Crying Kidneys, Cryocrystalglobulinemia, Cryoglobuinemia, Crystalglobulin-Induced Nephropathy, Crystal-Storing Histiocytosis, Cystic Kidney Disease, Acquired, Cystic Kidney Disease, Localized, Renal Failure, Cystinuria, Cystinosis, Dasatinib-Induced Nephrotic-Range Proteinuria, Deferasirox (Exjade) Nephrotoxicity, Dense Deposit Disease (MPGN Type 2), Dent Disease (X-linked Recessive Nephrolithiasis), DHA Crystalline Nephropathy, Dialysis Disequilibrium Syndrome, Diabetic Kidney Disease, Diffuse Mesangial Sclerosis, Diuresis, Djenkol Bean Poisoning (Djenkolism), Duplicated Ureter, EAST syndrome, Ectopic Kidney, Edema, Swelling, End-Stage Kidney Disease, Erdheim-Chester Disease, Fabry's Disease, Familial Hypocalciuric Hypercalcemia, Fanconi Syndrome, Fraser syndrome, Fibronectin Glomerulopathy, Fibrillary Glomerulonephritis and Immunotactoid Glomerulopathy, Fibrillary Glomerulonephritis, Congophilic, Fraley syndrome, Fluid Overload, Hypervolemia, Focal Segmental Glomerulosclerosis, Focal Sclerosis, Focal Glomerulosclerosis, Focal Segmental Glomerulosclerosis (Familial) with Complete Heart Block, Galloway Mowat syndrome, Giant Cell (Temporal) Arteritis with Kidney Involvement, Gestational Hypertension, Gitelman Syndrome, Glomerular Diseases, Glomerular Tubular Reflux, Glomerulonephritis, Glycosuria, Goodpasture Syndrome, Green Smoothie Cleanse Nephropathy, HANAC Syndrome, Harvoni (Ledipasvir with Sofosbuvir)-Induced Renal Injury, Hair Dye Ingestion and Acute Kidney Injury, Hantavirus Infection Podocytopathy, Heat Stress Nephropathy, Hematuria (Blood in Urine), Hemolytic Uremic Syndrome (HUS), Atypical Hemolytic Uremic Syndrome (aHUS), Hemophagocytic Syndrome, Hemorrhagic Cystitis, Hemorrhagic Fever with Renal Syndrome (HERS, Hantavirus Renal Disease, Korean Hemorrhagic Fever, Epidemic Hemorrhagic Fever, Nephropathis Epidemica), Hemosiderinuria, Hemosiderosis related to Paroxysmal Nocturnal Hemoglobinuria and Hemolytic Anemia, Hepatic Glomerulopathy, Hepatic Veno-Occlusive Disease, Sinusoidal Obstruction Syndrome, Hepatitis C-Associated Renal Disease, Hepatocyte Nuclear Factor 1β-Associated Kidney Disease, Hepatorenal Syndrome, Herbal Supplements and Kidney Disease, High Altitude Renal Syndrome, High Blood Pressure and Kidney Disease, HIV-Associated Immune Complex Kidney Disease (HIVICK), HIV-Associated Nephropathy (HIVAN), Holocaust Kidney Disease, HNFIB-related Autosomal Dominant Tubulointerstitial Kidney Disease, Horseshoe Kidney (Renal Fusion), Hunner's Ulcer, Hydrophilic Polymer Emboli, Hydroxychloroquine-induced Renal Phospholipidosis, Hyperaldosteronism, Hypercalcemia, Hyperkalemia, Hypermagnesemia, Hypernatremia, Hyperoxaluria, Hyperphosphatemia, Hypocalcemia, Hypocomplementemic Urticarial Vasculitic Syndrome, Hypokalemia, Hypokalemia-induced renal dysfunction, Hypokalemic Periodic Paralysis, Hypomagnesemia, Hyponatremia, Hypophosphatemia, Hypertension, Hypertension, Monogenic, Iced Tea Nephropathy, Ifosfamide Nephrotoxicity, IgA Nephropathy, IgG4 Nephropathy, Immersion Diuresis, Immune-Checkpoint Therapy-Related Interstitial Nephritis, Infliximab-Related Renal Disease, Interstitial Cystitis, Painful Bladder Syndrome, Interstitial Nephritis, Interstitial Nephritis, Karyomegalic, Ivemark's syndrome, JC Virus Nephropathy, Joubert Syndrome, Ketamine-Associated Bladder Dysfunction, Kidney-Based Immune Disorders, Kidney Stones, Nephrolithiasis, Kidney Stones due to Light Chains, Kimura Disease, Kombucha Tea Toxicity, Lead Nephropathy and Lead-Related Nephrotoxicity, Lecithin Cholesterol Acyltransferase Deficiency (LCAT Deficiency), Leptospirosis Renal Disease, Light Chain Deposition Disease, Monoclonal Immunoglobulin Deposition Disease, Light Chain Proximal Tubulopathy, Liddle Syndrome, Lightwood-Albright Syndrome, Lipoprotein Glomerulopathy, Lithium Nephrotoxicity, LMX1B Mutations Cause Hereditary FSGS, Loin Pain Hematuria, Lupus, Systemic Lupus Erythematosis, Lupus Kidney Disease, Lupus Nephritis, Lupus Nephritis with Antineutrophil Cytoplasmic Antibody Seropositivity, Lupus Podocytopathy, Lupus-like Nephritis, Lupus Nephritis, Lyme Disease-Associated Glomerulonephritis, Lysinuric Protein Intolerance, Lysozyme Nephropathy, Malarial Nephropathy, Malignancy-Associated Renal Disease, Malignant Hypertension, Malakoplakia, Marfan Syndrome and Kidney Disease, Mckittrick-Wheelock Syndrome, Mcatal Stenosis, Medullary Cystic Kidney Disease, Urolodulin-Associated Nephropathy, Juvenile Hyperuricemic Nephropathy Type 1, Medullary Sponge Kidney, Megaureter, MELAS Syndrome, Membranoproliferative Glomerulonephritis, Membranous Nephropathy, Membranous Nephropathy with Spherules, Membranous-like Glomerulopathy with Masked IgG Kappa Deposits, MesoAmerican Nephropathy, Metabolic Acidosis, Metabolic Alkalosis, Methotrexate-related Renal Failure, Microscopic Polyangiitis, Milk-alkalai syndrome, Minimal Change Discase, Monoclonal Gammopathy of Renal Significance, Dysproteinemia, Mouthwash Toxicity, MUC1 Nephropathy, Multicystic dysplastic kidney, Multiple Myeloma, Myeloproliferative Neoplasms and Glomerulopathy, Nail-patella Syndrome, NARP Syndrome, Nephrocalcinosis, Nephrocystin-1 Gene Deletions and ESRD, Nephrogenic Systemic Fibrosis, Nephronophthisis due to Nephrocystin-1 Gene Deletions, Nephroptosis (Floating Kidney, Renal Ptosis), Nephrotic Syndrome, Neurogenic Bladder, Nodular Glomerulosclerosis, Non-Gonococcal Urethritis, Nutcracker syndrome, Oligomeganephronia, Orofaciodigital Syndrome, Orotic Aciduria, Orthostatic Hypotension, Orthostatic Proteinuria, Osmotic Diuresis, Osmotic Nephrosis, Ovarian Hyperstimulation Syndrome, Oxalate Nephropathy, Page Kidney, Papillary Necrosis, Papillorenal Syndrome (Renal-Coloboma Syndrome, Isolated Renal Hypoplasia), The Peritoneal-Renal Syndrome, POEMS Syndrome, Posterior Urethral Valve, Podocyte Infolding Glomerulopathy, Post-infectious Glomerulonephritis, Post-streptococcal Glomerulonephritis, Post-infectious Glomerulonephritis, Atypical, Post-Infectious Glomerulonephritis (IgA-Dominant), Mimicking IgA Nephropathy, Polyarteritis Nodosa, Polycystic Kidney Disease, Posterior Urethral Valves, Post-Obstructive Diuresis, Preeclampsia, Propofol infusion syndrome, Proliferative Glomerulonephritis with Monoclonal IgG Deposits (Nasr Disease), Propolis (Honeybee Resin) Related Renal Failure, Proteinuria (Protein in Urine), Pseudohyperaldosteronism, Pseudohypobicarbonatemia, Pseudohypoparathyroidism, Pseudoporphyria, Psoriasis and Kidney Disease, Pulmonary-Renal Syndrome, Purple Urine Bag Syndrome, Pyelonephritis (Kidney Infection), Pyonephrosis, Radiation Nephropathy, Refeeding syndrome, Reflux Nephropathy, Rapidly Progressive Glomerulonephritis, REN Mutations Causing Autosomal Dominant Tubulointerstitial Kidney Disease, Renal Abscess, Peripnephric Abscess, Renal Agenesis, Renal Arcuate Vein Microthrombi-Associated Acute Kidney Injury, Renal Artery Aneurysm, Renal Artery Dissection, Spontaneous, Renal Artery Stenosis, Renal Cell Cancer, Renal Cyst, Renal Hypouricemia with Exercise-induced Acute Renal Failure, Renal Infarction, Renal Osteodystrophy, Renal Tubular Acidosis, Renin Mutations and Autosomal Dominant Tubulointerstitial Kidney Disease, Renin Secreting Tumors (Juxtaglomerular Cell Tumor), Reset Osmostat, Retrocaval Ureter, Retroperitoneal Fibrosis, Rhabdomyolysis, Rhabdomyolysis related to Bariatric Surgery, Rheumatoid Arthritis-Associated Renal Disease, Rubraca (rucaparib)-related Increase in Creatinine, Sarcoidosis Renal Disease, Saturday Night Acute Kidney Injury, Schistosomiasis and Glomerular Disease, Schimke immuno-osseous dysplasia, Scleroderma Renal Crisis, Serpentine Fibula-Polycystic Kidney Syndrome, Exner Syndrome, Sézary Syndrome, Sickle Cell Nephropathy, Sjögren's Syndrome and Renal Disease, Sri Lankan Farmers' Kidney Disease, Star Fruit Nephrotoxicity, renal transplantation, Kidney Disease Following Hematopoietic Cell Transplantation, Kidney Disease Related to Stem Cell Transplantation, TAFRO Syndrome, Tea and Toast Hyponatremia, Tenofovir-Induced Nephrotoxicity, Thin Basement Membrane Disease, Benign Familial Hematuria, Thrombotic Microangiopathy Associated with Monoclonal Gammopathy, Trench Nephritis, Autosomal Dominant Thrombotic Microangiopathy and CKD, Trigonitis, Tuberous Sclerosis, Tuberous sclerosis complex (TSC) and autosomal dominant polycystic kidney disease (ADPKD) (TSC2/PKD1 contiguous gene syndrome), Tubular Dysgenesis, Immune Complex Tubulointerstitial Nephritis, Tumor Lysis Syndrome, Uremia, Uremic Optic Neuropathy, Ureteritis Cystica, Ureterocele, Urethral Caruncle, Urethral Stricture, Urinary Incontinence, Urinary Tract Infection, Urinary Tract Obstruction, Urogenital Fistula, Vasomotor Nephropathy, Vesicointestinal Fistula, Vesicoureteral Reflux, Von Hippel-Lindau Disease, Waldenstrom's Macroglobulinemic Glomerulonephritis, Wegener's Granulomatosis, Granulomatosis with Polyangiitis, Wunderlich syndrome, Zellweger Syndrome, and Cerebrohepatorenal Syndrome.

### Pharmaceutical Compositions and Methods of Administration

To treat any of the foregoing disorders, pharmaceutical compositions for use in accordance with the methods of the present disclosure may be formulated in a conventional manner using one or more physiologically acceptable carriers. Pharmaceutically acceptable carriers are determined in part by the particular composition being administered, as well as by the particular method used to administer the composition. Accordingly, there are a wide variety of suitable formulations of the compounds useful in the methods of the present disclosure (see, e.g., Remington: The Science and Practice of Pharmacy, 20th ed., Gennaro et al. Eds., Lippincott Williams and Wilkins, 2000).

Formulations suitable for oral administration include, for example, solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives.

According to the present disclosure the compounds can be administered by any suitable means, which can vary, depending on the type of disorder being treated and on the nature of the compound itself. For example, the compounds may be administered orally, parenterally, or topically. For proteins such as antibodies, administration routes preferably include parenteral, e.g., intramuscular, intravenous, intraarterial, intraperitoneal, or subcutaneous. Preferably, the parenteral dosing is given by injection, most preferably intravenous, intramuscular, or subcutaneous injection. The amount to be administered will depend on a variety of factors such as the clinical symptoms, weight of the individual, and whether other drugs are administered. It should be appreciated that determination of proper dosage forms, dosage amounts, and routes of administration is within the level of ordinary skill in the pharmaceutical and medical arts, and is described below.

In one exemplary pharmaceutical composition containing the anti-CD40L antibody or antigen-binding fragment thereof, the composition is formulated as a sterile, preservative-free solution of the anti-CD40L antibody or antigen-binding fragment thereof for intravenous or subcutaneous administration. The formulation may be supplied as either a single-use, prefilled pen, as a single-use, for example containing about 1 mL prefilled glass syringe, or as a single-use institutional use vial. Preferably, the pharmaceutical composition containing the anti-CD40L antibody or antigen-binding fragment thereof is clear and colorless, with a pH of about 5.0 to about 6.9, preferably a pH of about 5.0 to about 6.5, and even more preferably a pH ranging from about 5.0 to about 6.0. In various embodiments, the formulations comprising the pharmaceutical compositions may contain from about 500 mg to about 1 mg, or from about 400 mg to about 10 mg, or from about 300 mg to about 30 mg or from about 200 mg to about 50 mg of the anti-CD40L antibody or antigen-binding fragment thereof per mL of solution when reconstituted and administered to the subject.

Selecting an administration regimen for a therapeutic depends on several factors, including the serum or tissue turnover rate of the entity, the level of symptoms, the immunogenicity of the entity, and the accessibility of the target cells in the biological matrix, general health of the patient, the prior medical history of the patient, and the like. Preferably, an administration regimen maximizes the amount of therapeutic delivered to the patient consistent with an acceptable level of side effects. Accordingly, the amount of biologic delivered depends in part on the particular entity and the severity of the condition being treated. It should be appreciated that determination of proper dosage forms, dosage amounts, and routes of administration is within the level of ordinary skill in the pharmaceutical and medical arts.

### Doses and Exposure Levels

The pharmaceutical formulations of the present disclosure may contain from about 0.001 to about 200 mg/kg of an anti-CD40L antibody or antigen binding fragment thereof, for example, from about 0.001 mg/kg to about 100 mg/kg, or from about 0.001 mg/kg to about 50 mg/kg, or from about 0.001 mg/kg to about 10 mg/kg intravenous injection of the anti-CD40L antibody, or antigen-binding fragment thereof, may be given as a bolus, and the rest of the antibody dose may be administered by intravenous injection. A predetermined dose of the anti-CD40L antibody, or antigen-binding fragment thereof, may be administered, for example, over a period of an hour to two hours to five hours.

In a further embodiment, part of the dose is administered by a subcutaneous injection and/or infusion in the form of a bolus and the rest by infusion of the antibody formulation. In some exemplary doses, the antibody formulation may be administered subcutaneously in a dose ranging from about 0.001 to about 200 mg/kg, for example, from about 0.001 mg/kg to about 100 mg/kg, or from about 0.001 mg/kg to about 50 mg/kg, or from about 0.001 mg/kg to about 10 mg/kg intravenous injection of the anti-CD40L antibody, or antigen-binding fragment thereof. In some embodiments the dose may be given as a bolus, and the rest of the antibody dose may be administered by subcutaneous or intravenous injection. A predetermined dose of the anti-CD40L antibody, or antigen-binding fragment thereof, may be administered, for example, over a period of an hour, or a period of two hours, or a period of three hours, or a period of four hours or a period of five hours or longer.

### Combination Therapies

The antibodies or antibody fragments thereof described herein can be administered alone (monotherapy) or in combination, i.e., combined with other agents. For example, in one embodiment the combination therapy may include one or more additional therapeutic agents. In another embodiment the combination therapy includes standard of care treatment that may, or may not, include additional therapeutic agents (consists essentially of the antibody or antibody fragment thereof).

Adjunctive or combined administration (co-administration) includes simultaneous administration of any of the antibodies or antigen binding fragments thereof, described herein and one or more agents in the same or different dosage form, or separate administration of the polypeptide and one or more agents (e.g., sequential administration). Such concurrent or sequential administration preferably results in both the polypeptide and the one or more agents being simultaneously present in treated patients.

### Kits and Articles of Manufacture

Further provided are kits containing the antibody or antigen binding fragments thereof described herein and instructions for use. Kits typically include a packaged combination of reagents in predetermined amounts with instructions and a label indicating the intended use of the contents of the kit. The term label or instruction includes any writing, or recorded material supplied on or with the kit, or which otherwise accompanies the kit at any time during its manufacture, transport, sale, or use. It can be in the form prescribed by a governmental agency regulating the manufacture, use or sale of pharmaceuticals or biological products, which notice reflects approval by the agency of the manufacture, use or sale for administration to a human or for veterinary use. The label or instruction can also encompass advertising leaflets and brochures, packaging materials, and audio or video instructions.

### Anti-CD40L Antigen Binding Molecules

Some embodiments provided herein relate to antibodies that are modified versions of the anti-CD40L antibody hu5c8 that comprise a human IgG1 consensus framework having the variable light chain and the variable heavy chain CDR sequences of hu5c8 with an Fc domain modified to prevent platelet activation.

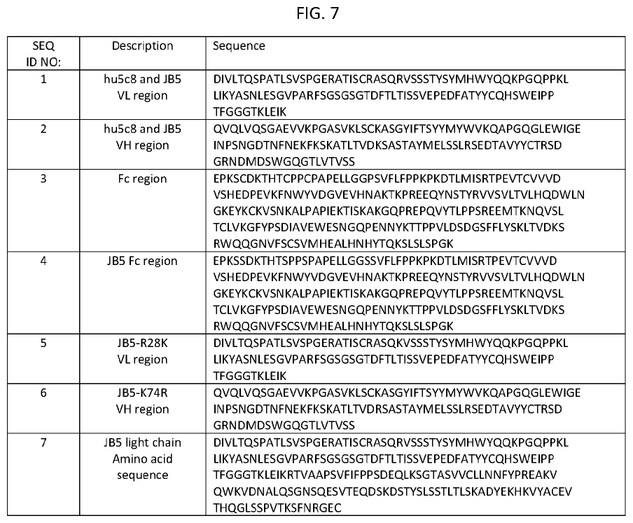

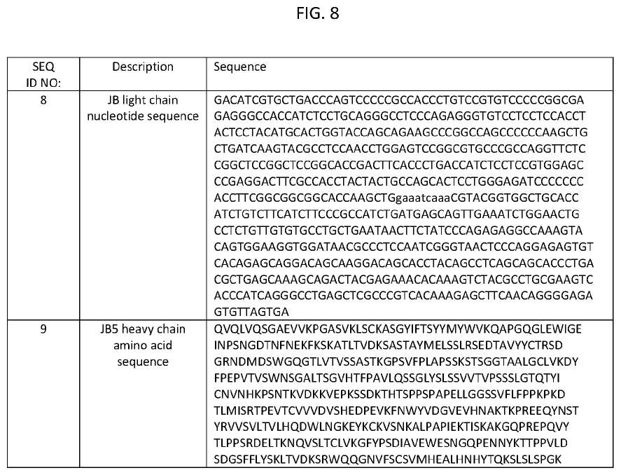

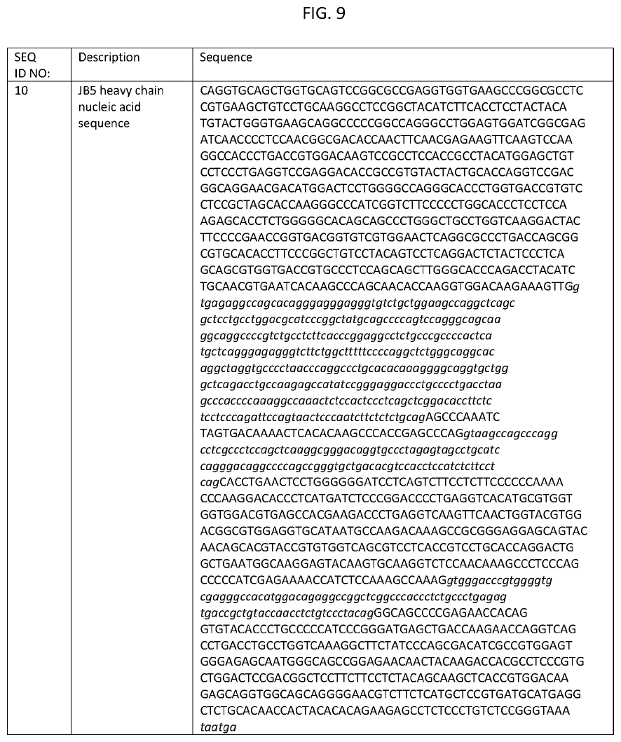

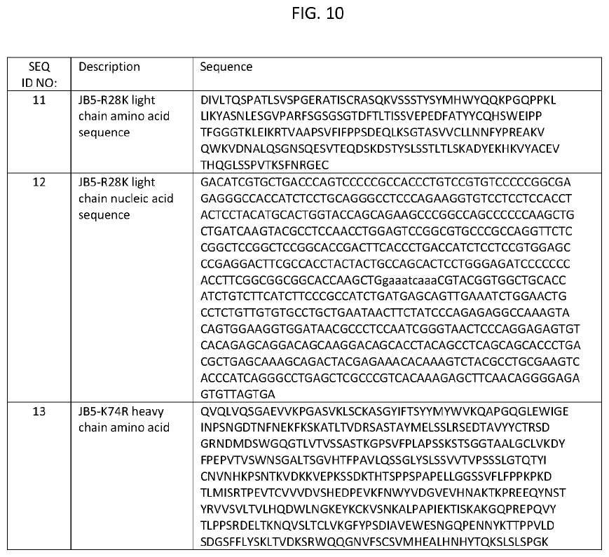

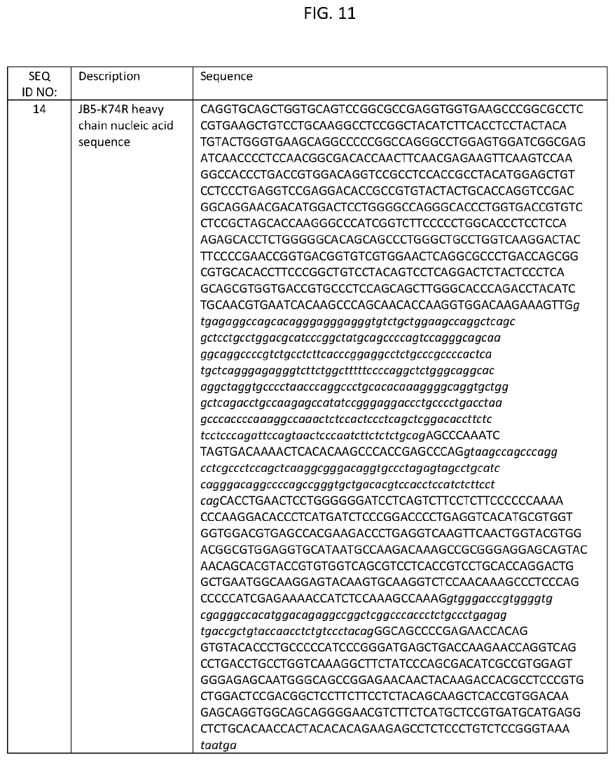

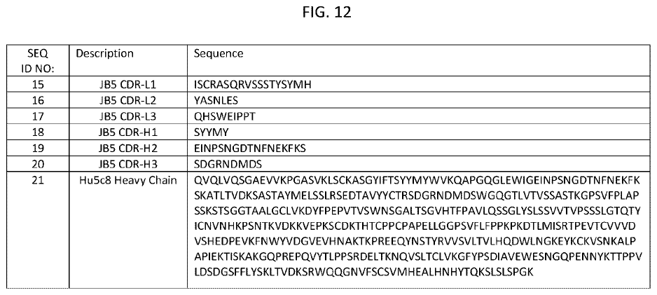

Table 3 provides a description of the SEQ ID NOs: referenced in the application (and shown in FIGS. 7-12).

TABLE 3

| SEQ | |
|---|---|
| ID | |
| NO: | Description of Sequence |
| 1 | Light chain variable region amino acid sequence (hu5c8 and JB5) |
| 2 | Heavy chain variable region amino acid sequence (hu5c8 and JB5) |
| 3 | Fc region amino acid sequence (hu5c8) |
| 4 | JB5 Fc region amino acid sequence |
| 5 | JB5-R28K light chain variable region amino acid sequence |
| 6 | JB5-K74R heavy chain variable region amino acid sequence |
| 7 | JB5 light chain amino acid sequence |
| 8 | JB5 light chain nucleic acid sequence |
| 9 | JB5 heavy chain amino acid sequence |
| 10 | JB5 heavy chain nucleic acid sequence |
| 11 | JB5-R28K light chain amino acid sequence |
| 12 | JB5-R28K light chain synthetic gene nucleic acid sequence |
| 13 | JB5-K74R heavy chain amino acid sequence |
| 14 | JB5-K74R heavy chain synthetic gene nucleic acid sequence |
| 15 | CDR-1 of the JB5 Variable Light Chain amino acid sequence |
| 16 | CDR-2 of the JB5 Variable Light Chain amino acid sequence |
| 17 | CDR-3 of the JB5 Variable Light Chain amino acid sequence |
| 18 | CDR-1 of the JB5 Variable Heavy Chain amino acid sequence |
| 19 | CDR-2 of the JB5 Variable Heavy Chain amino acid sequence |
| 20 | CDR-3 of the JB5 Variable Heavy Chain amino acid sequence |
| 21 | Hu5c8 Heavy Chain amino acid sequence |

One embodiment (embodiment A) is an isolated antibody that binds to CD40L and that comprises a light chain and a heavy chain, wherein the light chain comprises a light chain variable region comprising an amino acid sequence having at least 90%, or at least 91%, or at least 92%, or at least 93%, or at least 94%, or at least 95%, or at least 96% or at least 97%, or at least 98% or at least 99% sequence identity with SEQ ID NO: 1 and the heavy chain comprises a variable heavy chain region and an Fc region, wherein the heavy chain variable region comprises an amino acid sequence having at least 90%, or at least 91%, or at least 92%, or at least 93%, or at least 94%, or at least 95%, or at least 96%, or at least 97%, or at least 98%, or at least 99% sequence identity with SEQ ID NO: 2 and the Fc region comprises an amino acid sequence having at least at least 90%, or at least 91%, or at least 92%, or at least 93%, or at least 94%, or at least 95%, or at least 96%, or at least 97%, or at least 98%, or at least 99% sequence identity with SEQ ID NO: 3 wherein the Fc region comprises one or a combination of substitutions selected from the group consisting of C11S, C14S, and P23S.

Another embodiment (embodiment B) is an isolated antibody according to embodiment A, wherein the Fc region further comprises the amino acid substitution C5S.

In variations of the embodiments A and B the antibody comprises a light chain variable region that does not comprise any of the substitutions T33W, S26D, and Q27E.

In other variations of embodiments A and B, the light chain variable region comprises the substitution R28K.

In some variations of the embodiments of A and B, the CDRs of the heavy and light chain have the sequences listed in Table 4.

TABLE 4

| | |
|---|---|
| CDR1 light chain | ISCRASQRVSSSTYSYMH |
| | (SEQ ID NO: 15) |
| CDR2 light chain | YASNLES (SEQ ID NO: 16) |
| CDR3 light chain | QHSWEIPPT (SEQ ID NO: 17) |
| CDR1 heavy chain | SYYMY (SEQ ID NO: 18) |
| CDR2 heavy chain | EINPSNGDTNFNEKFKS |
| | (SEQ ID NO: 19) |
| CDR3 heavy chain | SDGRNDMDS (SEQ ID NO: 20) |

In yet other variation of embodiments A and B, the light chain variable region comprises the amino acid sequence ICRRASQRVSSSTYSYMH (SEQ ID NO: 15). In still other embodiments, the light chain variable region comprises the amino acid sequence ICRRASQRVSSSTYSYMH (SEQ ID NO: 15) and one or both of the amino acid sequences YASNLES (SEQ ID NO: 16) and QHSWEIPPT (SEQ ID NO: 17).

In some variations of embodiments A and B, the light chain variable region comprises the amino acid sequence of SEQ ID NO: 1. In yet other embodiments the light chain variable region consists of the amino acid of SEQ ID NO: 1. In some embodiments, the light chain consists essentially of the amino acid sequence of SEQ ID NO: 7. In other embodiments, the light chain consists of the amino acid sequence of SEQ ID NO: 7. In still other embodiments, the light chain comprises the amino acid sequence of SEQ ID NO: 11. In yet other embodiments, the light chain consists essentially of the amino acid sequence of SEQ ID NO: 11. In still other embodiments, the light chain consists of the amino acid sequence of SEQ ID NO: 11.

In other variations of the embodiments A and B, the antibody comprises a heavy chain variable region that does not comprise any of the substitutions T30H, Y33W, or S54N. In some embodiments of the antibodies of embodiments A and B, the light chain variable region does not comprise any of the substitutions T33W, S26D, and Q27E. In other variations of embodiments A and B, the light chain variable region does not comprise any of the substitutions T33W, S26D, and Q27E and the heavy chain variable region does not comprise any of the substitutions T30H, Y33W, or S54N.

In yet other variations of the embodiments A and B, the heavy chain variable region comprises the substitution K74R. In one embodiment the heavy chain variable region comprises one or any combination of the amino acid sequences SYYMY (SEQ ID NO: 18), EINPSNGDTNFNEKFKS (SEQ ID NO: 19), and SDGRNDMDS (SEQ ID NO: 20).

In another embodiment, the heavy chain variable region comprises the amino acid sequence of SEQ ID NO: 2. In yet another embodiment the heavy chain variable region consists essentially of the amino acid sequence of SEQ ID NO: 2. In still another embodiment the heavy chain variable region consists of the amino acid sequence of SEQ ID NO: 2. In some embodiments, the heavy chain variable region comprises the amino acid sequence of SEQ ID NO: 6. In yet other embodiments the heavy chain variable region consists essentially of the amino acid sequence of SEQ ID NO: 6. In still other embodiments the heavy chain variable region consists of the amino acid sequence of SEQ ID NO: 6.

Some embodiments provided herein relate to an isolated antibody, wherein the light chain comprises the amino acid sequence of SEQ ID NO: 1 and the heavy chain consists of the amino acid sequence of SEQ ID NO: 9.

Some embodiments provided herein relate to an isolated antibody, wherein the light chain consists of the amino acid sequence of SEQ ID NO: 7 and the heavy chain consists of the amino acid sequence of SEQ ID NO: 9.

Some embodiments provided herein relate to an isolated antibody wherein the light chain variable region comprises the amino acid sequence of SEQ ID NO: 5 and the heavy chain consists of the amino acid sequence of SEQ ID NO: 9.

Still another embodiment is an isolated antibody wherein the light chain consists of the amino acid sequence of SEQ ID NO: 11 and the heavy chain consists of the amino acid sequence of SEQ ID NO: 9.

Yet another embodiment, is an isolated antibody wherein the light chain consists of the amino acid sequence of SEQ ID NO: 7 and the heavy chain consists of the amino acid sequence of SEQ ID NO: 13.

Another embodiment is an isolated antibody wherein the light chain consists of the amino acid sequence of SEQ ID NO: 11 and the heavy chain consists of the amino acid sequence of SEQ ID NO: 13.

Some embodiments of the present application relate to modifying biomarkers in a subject with treatment of a compound targeting CD40/CD40L. In some embodiments, the biomarker is a protein involved in CD40/CD40L activity. In some embodiments, the biomarker is CD40. In some embodiments, the biomarker is CD40L. In some embodiments, the biomarker is NFL. In some embodiments, the biomarker is a pro-inflammatory marker, or linked to inflammation. In some embodiments, the biomarker is a cytokine and/or an interleukin. In some embodiments, the biomarker is one or more of IgA, IgE, CD40L, C3, CXCL9, CXCL10, and/or IgM.

In some embodiments, treatment with a compound targeting CD40/CD40L increases the concentration of one or more biomarkers. In some embodiments, treatment with a compound targeting CD40/CD40L decreases the concentration of one or more biomarkers. In some embodiments, treatment with a compound targeting CD40/CD40L increases at least one biomarker concentration, and decreases at least one other biomarker concentration. In some embodiments, the baseline levels of the at least one biomarker serve as a predictor for the response of a subject with a kidney-related disease, disorder, or condition to treatment with a compound targeting CD40/CD40L.

Some embodiments provided herein are further described in the following enumerated alternatives.

1. A method of predicting the response of a subject with a kidney-related disease or disorder to treatment with a compound against CD40L or CD40, the method comprising:
collecting a sample from the subject;
measuring a concentration of at least one biomarker present in the sample, wherein the at least one biomarker is CXCL9, CXCL10, IgM, C3, IgA, IgE, CD40L, and CD40;
wherein the concentration of the at least one biomarker is predictive that the subject is likely to be responsive to treatment of the kidney-related disease or disorder with a compound against CD40L or CD40.

2. The method of alternative 1, wherein the kidney-related disease or disorder comprises inflammation.

3. The method of any one of the preceding alternatives, wherein the subject is mammalian and/or human.

4. The method of any one of the preceding alternatives, wherein the compound blocks the interaction between CD40 and CD40L.

5. The method of any one of the preceding alternatives, wherein the compound is an anti-CD40L antibody or an anti-CD40 antibody.

6. The method of any one of the preceding alternatives, wherein the compound is AT-1501 or 5c8.

7. The method of any one of the preceding alternatives, wherein the compound is MR1.

8. The method of any one of the proceeding alternatives, wherein the kidney-related disease or disorder is selected from the group consisting of colitis, drug induced lupus nephritis, lupus nephritis, nephropathy, chronic kidney disease, graft versus host disease, immune graft response, kidney transplant, transplant rejection, atherosclerosis, diabetic kidney disease, acute kidney injury, kidney failure, polycystic kidney disease, hematuria, Fabry disease, lupus kidney disease, Alport's syndrome, IgM nephropathy, interstitial nephritis, Membranoproliferative Glomerulonephritis, simple kidney cysts, kidney infections, kidney stones, and IgA nephropathy.

9. The method of any one of the proceeding alternatives, wherein the kidney-related disease or disorder is an autoimmune disease, wherein the autoimmune disease is systemic lupus erythematous, type-1 diabetes, myasthenia gravis, inflammatory bowel disease, immune thrombocytopenia purpura, rheumatoid arthritis, psoriasis, Addison's disease, Crohn's disease, uveitis, multiple sclerosis, hemolytic anemia, or Graves' disease.

10. The method of any one of the proceeding alternatives, wherein the sample is blood, urine, saliva, or mucus.

11. The method of alternative 10, wherein the sample is urine.

12. A method treating a subject with a kidney-related disease or disorder, the method comprising: administering a therapeutically effective dose of a compound against CD40L or CD40.

13. The method of alternative 12, wherein the kidney-related disease or disorder comprises inflammation.

14. The method of any one of alternatives 12-13, in which the subject is mammalian and/or human.

15. The method of any one of alternatives 12-14, wherein the compound blocks the interaction between CD40 and CD40L.

16. The method of any one of alternatives 12-15, wherein the compound is an anti-CD40L antibody or an anti-CD40 antibody.

17. The method of any one of alternatives 12-16, wherein the compound is AT-1501.

18. The method of any one of alternatives 12-17, wherein the compound is MR1 or 5c8.

19. The method of any one of alternatives 12-18, wherein the compound is administered at least once every three weeks.

20. The method of any one of alternatives 12-19, wherein the compound is administered at a dose of between 200 mg/kg and 100 mg/kg; preferably at a dose between 1 mg/kg and 20 mg/kg.

21. The method of any one of alternatives 12-20, wherein the kidney-related disease or disorder is colitis, drug induced lupus nephritis, lupus nephritis, nephropathy, chronic kidney disease, graft versus host disease, immune graft response, transplant rejection, atherosclerosis, diabetic kidney disease, acute kidney injury, kidney failure, polycystic kidney disease, hematuria, Fabry disease, lupus kidney disease, Alport's syndrome, IgM nephropathy, interstitial nephritis, Membranoproliferative Glomerulonephritis, simple kidney cysts, kidney infections, kidney stones, or IgA nephropathy.

22. The method of any one of alternatives 12-21, wherein the kidney-related disease or disorder is an autoimmune disease, wherein the autoimmune disease is systemic lupus erythematous, type-1 diabetes, myasthenia gravis, inflammatory bowel disease, immune thrombocytopenia purpura, rheumatoid arthritis, psoriasis, Addison's disease, Crohn's disease, uveitis, multiple sclerosis, hemolytic anemia, or Graves' disease.

23. The method of any one of alternatives 12-22, the method further comprising administering a second pharmaceutically effective compound.

24. The method of alternative 23, wherein the second compound blocks the interaction between CD28 and CD86 or between CD28 and CD80.

25. The method of alternative 23, wherein the second compound targets at least one of the biomarkers selected from the group consisting of: interleukins, cytokines, and proinflammatory markers.

26. The method of alternative 23, wherein the second compound is a CTLA4-Ig fusion protein, an abatacept, a belatacept, or a galiximab.

27. The method of any one of alternatives 12-26, wherein the compound is administered orally, parenterally, or topically.

28. The method of alternative 27, wherein the compound is administered parenterally.

29. The method of alternative 28, wherein the compound is administered by injection, most preferably intravenous, intramuscular, or subcutaneous injection.

30. The method of any one of the proceeding alternatives, wherein the compound that targets CD40L or CD40 is an antibody that comprises:
(a) a heavy chain variable region (VH) comprising:
   i) a CDRH1 domain comprising the sequence set forth in SEQ ID NO: 9;
   ii) a CDRH2 domain comprising the sequence set forth in SEQ ID NO: 10, 11, 12, 13, or 14; and
   iii) a CDRH3 domain comprising the sequence set forth in SEQ ID NO: 15; and
(b) a light chain variable region (VL) comprising:
   i) a CDRL1 domain comprising the sequence set forth in SEQ ID NO: 16 or 17;
   ii) a CDRL2 domain comprising the sequence set forth in SEQ ID NO: 18 or 19; and
   iii) a CDRL3 domain comprising the sequence set forth in SEQ ID NO: 20.

31. The method of any one of the proceeding alternatives, wherein the compound that targets CD40L or CD40 is an antibody that comprises:
a) a heavy chain variable region (VH) having the amino acid sequence as set forth in SEQ ID NOs: 1, 2, 3, or 4; and
b) a light chain variable region (VL) having the amino acid sequence as set forth in SEQ ID NOs: 5, 6, 7, or 8.

32. A method of modulating the concentration of at least one biomarker in a subject, the method comprising:
administering to the subject a therapeutically effective amount of a compound against CD40L or CD40,
wherein the biomarkers are selected from the group consisting of: IgA, IgE, CD40L, CD40, C3, CXCL9, CXCL10, and IgM.

33. The method of alternative 32, wherein the concentration of at least one biomarker increases following administration of the compound.

34. The method of alternative 32, wherein the concentration of at least one biomarker decreases following administration of the compound.

35. The method of alternative 32, wherein the concentration of at least one biomarker increases, and the concentration of at least one other biomarker decreases, following administration of the compound.

36. The method of any one of alternatives 32-35, wherein the subject has a kidney-related disease or disorder.

37. The method of alternative 36, wherein the kidney-related disease or disorder comprises inflammation.

38. The method of any one of alternatives 32-37, wherein the subject is mammalian and/or human.

39. The method of any one of alternatives 32-38, wherein the compound blocks the interaction between CD40 and CD40L.

40. The method of any one of alternatives 32-39, wherein the compound is an anti-CD40L antibody or an anti-CD40 antibody.

41. The method of any one of alternatives 32-40, wherein the compound is AT-1501.

42. The method of any one of alternatives 32-41, wherein the compound is MR1 or 5c8.

43. The method of any one of alternatives 36-42, wherein the kidney-related disease or disorder is colitis, drug induced lupus nephritis, lupus nephritis, nephropathy, chronic kidney disease, graft versus host disease, immune graft response, transplant rejection, atherosclerosis, diabetic kidney disease, acute kidney injury, kidney failure, polycystic kidney disease, hematuria, Fabry disease, lupus kidney disease, Alport's syndrome, IgM nephropathy, interstitial nephritis, Membranoproliferative Glomerulonephritis, simple kidney cysts, kidney infections, kidney stones, or IgA nephropathy.

44. The method of any one of alternatives 36-43, wherein the kidney-related disease or disorder is an autoimmune disease, wherein the autoimmune disease is systemic lupus erythematous, type-1 diabetes, myasthenia gravis, inflammatory bowel disease, immune thrombocytopenia purpura, rheumatoid arthritis, psoriasis, Addison's disease, Crohn's disease, uveitis, multiple sclerosis, hemolytic anemia, or Graves' disease.

45. The method of any one of alternatives 32-44, wherein the compound is administered at least once every three weeks.

46. The method of any one of alternatives 32-45, wherein the compound is administered at a dose of between 200 mg/kg and 100 mg/kg; preferably at a dose between 1 mg/kg and 20 mg/kg.

47. The method of any one of alternatives 32-46, wherein the compound is administered orally, parenterally, or topically.

48. The method of alternative 47, wherein the compound is administered parenterally.

49. The method of alternative 47, wherein the compound is administered by injection, most preferably intravenous, intramuscular, or subcutaneous injection.

50. The method of any one of alternatives 33-49, wherein the compound that targets CD40L or CD40 is an antibody that comprises:
a. a heavy chain variable region (VH) comprising:
i) a CDRH1 domain comprising the sequence set forth in SEQ ID NO: 9;
ii) a CDRH2 domain comprising the sequence set forth in SEQ ID NO: 10, 11, 12, 13, or 14; and
iii) a CDRH3 domain comprising the sequence set forth in SEQ ID NO: 15; and
b. a light chain variable region (VL) comprising:
i. a CDRL1 domain comprising the sequence set forth in SEQ ID NO: 16 or 17;
ii. a CDRL2 domain comprising the sequence set forth in SEQ ID NO: 18 or 19; and
iii. a CDRL3 domain comprising the sequence set forth in SEQ ID NO: 20.

51. The method of any one of alternatives 32-50, wherein the compound that targets CD40L or CD40 is an antibody that comprises:
a. a heavy chain variable region (VI) having the amino acid sequence as set forth in SEQ ID NOs: 1, 2, 3, or 4; and
b. a light chain variable region (VL) having the amino acid sequence as set forth in SEQ ID NOs: 5, 6, 7, or 8.

### EXAMPLES

Embodiments are further defined in the following Examples. It should be understood that these Examples are given by way of illustration only. From the above discussion and these Examples, one skilled in the art can ascertain the essential characteristics, and without departing from the spirit and scope thereof, can make various changes and modifications of the embodiments to adapt it to various usages and conditions. Thus, various modifications of the embodiments, in addition to those shown and described herein, will be apparent to those skilled in the art from the foregoing description. Such modifications are also intended to fall within the scope of the appended claims. The disclosure of each reference set forth herein is incorporated herein by reference in its entirety, and for the disclosure referenced herein.

### Example 1

Clinical Trials with Kidney Transplants

Four human subjects with IgAN (Berger's Disease) were given kidney transplants and monitored for a period of 12 weeks. At the end of those twelve weeks, a urine sample was collected from each subject. These samples were then screened for changes in proinflammatory markers, compared to levels in each subject's urine prior to the kidney transplant.

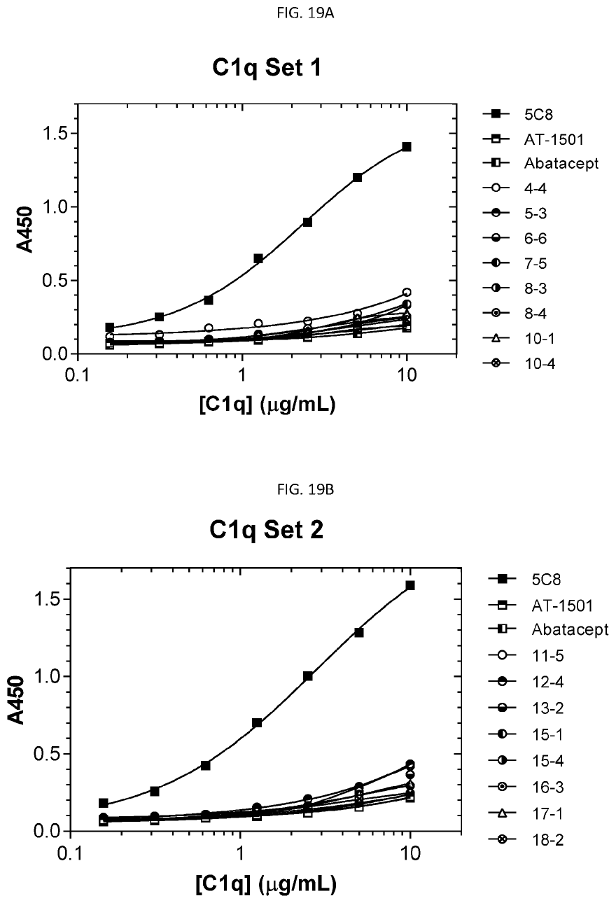

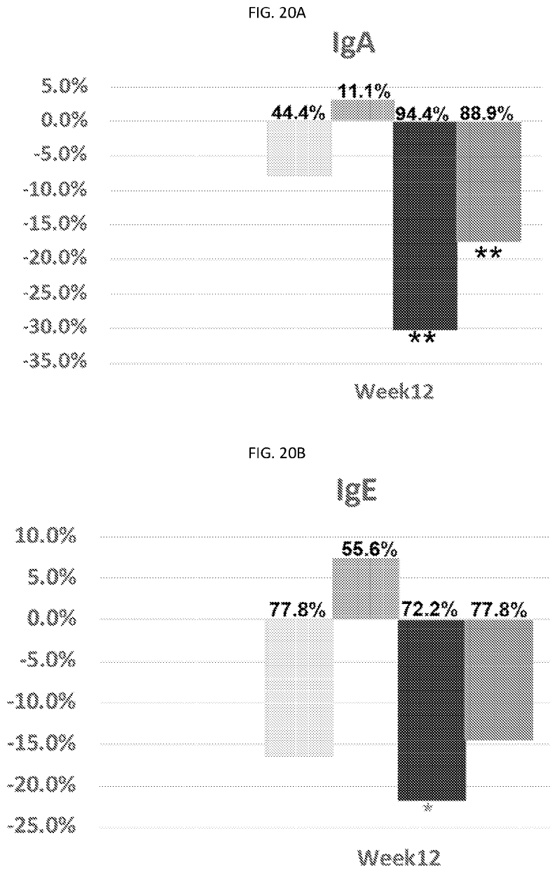

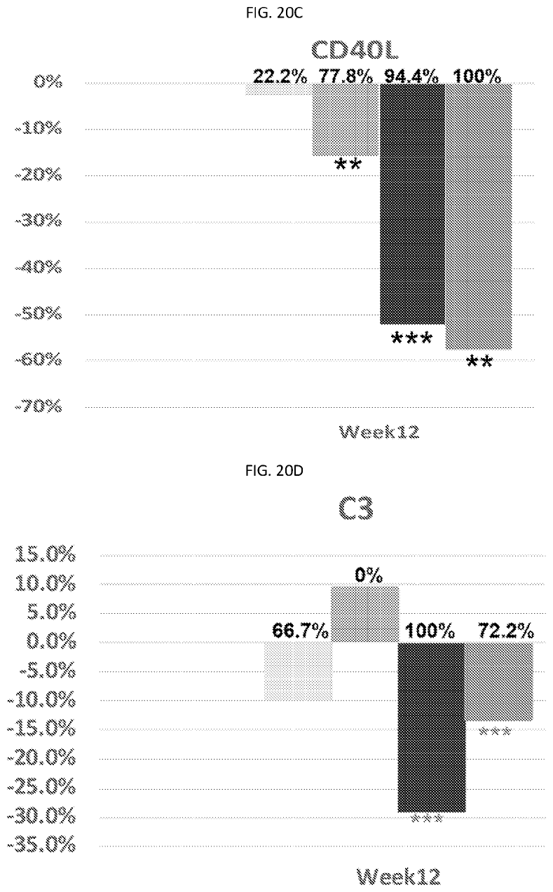

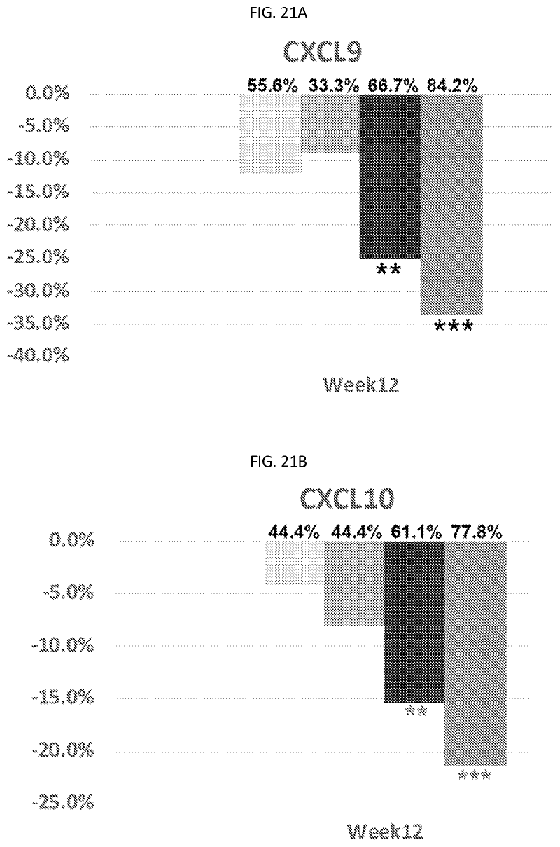

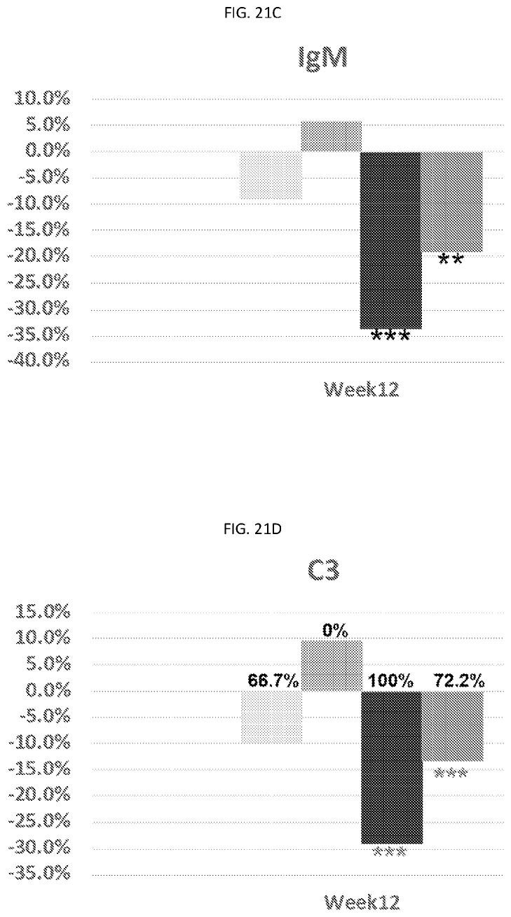

The IgAN-correlated proinflammatory markers IgA, IgE, CD40L, and C3 noticeably decreased in patients after 12 weeks (FIGS. 20A-20D). Furthermore, the general transplant proinflammatory markers CXCL9, CXCL10, IgM, and C3 decreased after 12 weeks (FIGS. 21A-21D). Given the strong decrease in CD40L and CD40L-correlated proteins in subjects upon transplanting with a healthy kidney, it appears likely that subsequent treatment with CD40L targeting compounds such as those disclosed herein may help accelerate recovery and the reduction of inflammation in subjects.

### Example 2

### Phase 1B Human Clinical Trials

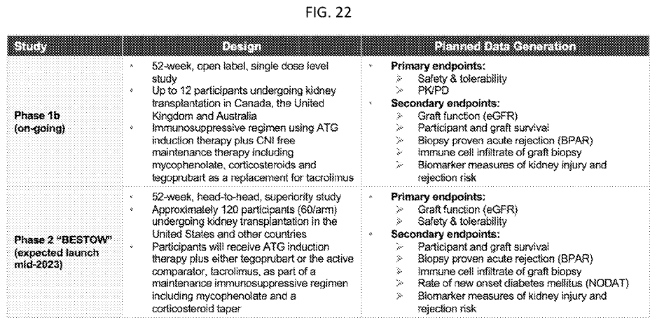

The clinical trials were expanded into a 52-week, open label, single dose level study of up to 12 participants. The details of the study design are as shown in FIG. 22. The participants are administered an immunosuppressive regimen using ATG induction therapy plus CNI free maintenance therapy, including mycophenolate, corticosteroids, and tegoprubart as a replacement for tacrolimus. The drugs were administered as follows:
rATG: 1.5 mg/kg/dose IV for 4 doses (total of 6 mg/kg). The first dose is started prior to revascularization and the remaining 3 doses (total 4.5 mg/kg) are within 5-10 days post-transplant consistent with labeled instructions.
Corticosteroid: Per standard of care, to be tapered to 5 mg prednisone daily (or equivalent) by 28 days post-transplant.
MMF: 1000 mg BID or MPS: 720 mg BID.
Tegoprubart: 20 mg/kg via IV infusion on Days 1, 3, 7, 14, 21, 28 and every 21 days thereafter.

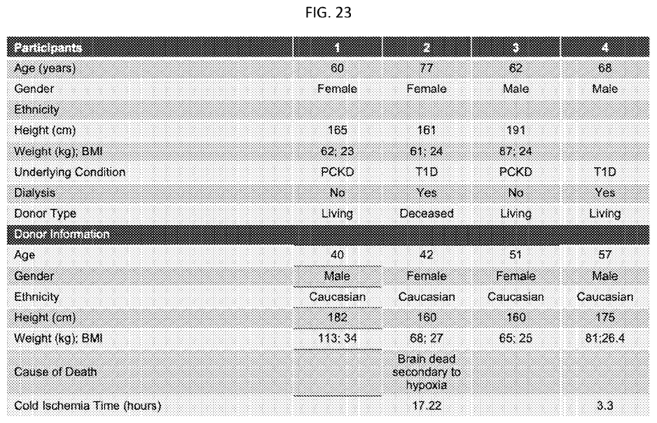

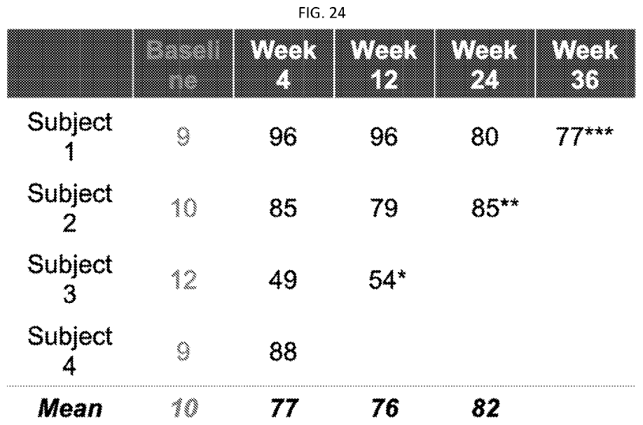

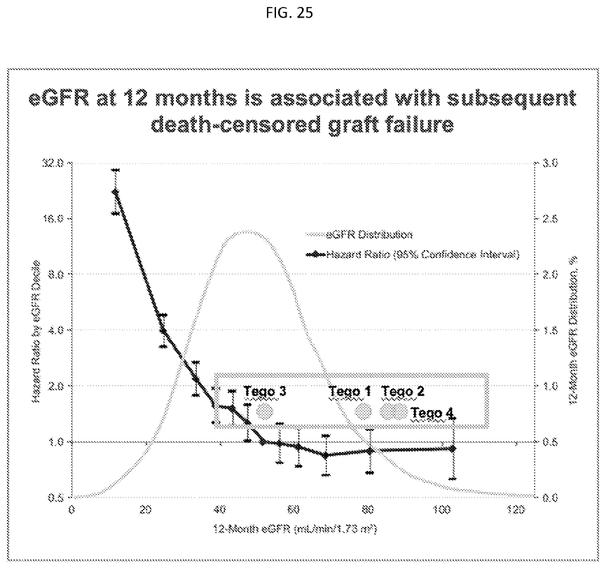

The first four participants selected for the trial were two females and two males, ages 60-77 (FIG. 23). These participants each underwent a kidney transplant followed by the dosing as described above. Kidney function was monitored for the next 120 using the estimated glomerular filtration rate (eGFR) (FIGS. 24-25). Tegoprubart was administered at four different times, as shown in FIG. 25. Thus far, kidney function has been greatly enhanced with a low hazard ratio.

As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose, including the disclosures specifically referenced herein. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

Although embodiments described herein have been disclosed in the context of certain embodiments and examples, those skilled in the art will understand that the present disclosure extends beyond the specifically disclosed embodiments to other alternative embodiments and/or uses of the disclosure and obvious modifications and equivalents thereof. In addition, while several variations of the embodiments have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes or embodiments. Thus, it is intended that the scope of the present disclosure should not be limited by the particular disclosed embodiments described above.

It should be understood, however, that this detailed description, while indicating preferred embodiments, is given by way of illustration only, since various changes and modifications within the spirit and scope of the disclosure will become apparent to those skilled in the art.

The terminology used in the description presented herein is not intended to be interpreted in any limited or restrictive manner. Rather, the terminology is simply being utilized in conjunction with a detailed description of embodiments of the systems, methods, and related components. Furthermore, embodiments may comprise several novel features, no single one of which is solely responsible for its desirable attributes or is believed to be essential to practicing the embodiments herein described.

What is claimed is:

1. A method of predicting the response of a subject with a kidney-related disease or disorder to treatment with a compound against CD40L or CD40, the method comprising:
collecting a sample from the subject;
measuring a concentration of at least one biomarker present in the sample, wherein the at least one biomarker is CXCL9, CXCL10, IgM, C3, IgA, IgE, CD40L, and CD40;
wherein the concentration of the at least one biomarker is predictive that the subject is likely to be responsive to treatment of the kidney-related disease or disorder with a compound against CD40L or CD40.

2. The method of claim 1, wherein the kidney-related disease or disorder comprises inflammation.

3. The method of claim 1, wherein the subject is mammalian and/or human.

4. The method of claim 1, wherein the compound blocks the interaction between CD40 and CD40L.

5. The method of claim 1, wherein the compound is an anti-CD40L antibody or an anti-CD40 antibody.

6. The method of claim 1, wherein the compound is AT-1501 or 5c8.

7. The method of claim 1, wherein the compound is MR1.

8. The method of claim 1, wherein the kidney-related disease or disorder is selected from the group consisting of colitis, drug induced lupus nephritis, lupus nephritis, nephropathy, chronic kidney disease, graft versus host disease, immune graft response, kidney transplant, transplant rejection, atherosclerosis, diabetic kidney disease, acute kidney injury, kidney failure, polycystic kidney disease, hematuria, Fabry disease, lupus kidney disease, Alport's syndrome, IgM nephropathy, interstitial nephritis, Membranoproliferative Glomerulonephritis, simple kidney cysts, kidney infections, kidney stones, and IgA nephropathy.

9. The method of claim 1, wherein the kidney-related disease or disorder is an autoimmune disease, wherein the autoimmune disease is systemic lupus erythematous, type-1 diabetes, myasthenia gravis, inflammatory bowel disease, immune thrombocytopenia purpura, rheumatoid arthritis, psoriasis, Addison's disease, Crohn's disease, uveitis, multiple sclerosis, hemolytic anemia, or Graves' disease.

10. The method of claim 1, wherein the sample is blood, urine, saliva, or mucus.

11. The method of claim 10, wherein the sample is urine.

12. A method treating a subject with a kidney-related disease or disorder, the method comprising:
administering a therapeutically effective dose of a compound against CD40L or CD40.

13. The method of claim 12, wherein the kidney-related disease or disorder comprises inflammation.

14. The method of claim 12, in which the subject is mammalian and/or human.

15. The method of claim 12, wherein the compound blocks the interaction between CD40 and CD40L.

16. The method of claim 12, wherein the compound is an anti-CD40L antibody or an anti-CD40 antibody.

17. The method of claim 12, wherein the compound is AT-1501.

18. The method of claim 12, wherein the compound is MR1 or 5c8.

19. The method of claim 12, wherein the compound is administered at least once every three weeks.

20. The method of claim 12, wherein the compound is administered at a dose of between 200 mg/kg and 100 mg/kg; preferably at a dose between 1 mg/kg and 20 mg/kg.

21. The method of claim 12, wherein the kidney-related disease or disorder is colitis, drug induced lupus nephritis, lupus nephritis, nephropathy, chronic kidney disease, graft versus host disease, immune graft response, transplant rejection, atherosclerosis, diabetic kidney disease, acute kidney injury, kidney failure, polycystic kidney disease, hematuria, Fabry disease, lupus kidney disease, Alport's syndrome, IgM nephropathy, interstitial nephritis, Membranoproliferative Glomerulonephritis, simple kidney cysts, kidney infections, kidney stones, or IgA nephropathy.

22. The method of claim 12, wherein the kidney-related disease or disorder is an autoimmune disease, wherein the autoimmune disease is systemic lupus erythematous, type-1 diabetes, myasthenia gravis, inflammatory bowel disease, immune thrombocytopenia purpura, rheumatoid arthritis, psoriasis, Addison's disease, Crohn's disease, uveitis, multiple sclerosis, hemolytic anemia, or Graves' disease.

23. The method of claim 12, the method further comprising administering a second pharmaceutically effective compound.

24. The method of claim 23, wherein the second compound blocks the interaction between CD28 and CD86 or between CD28 and CD80.

25. The method of claim 23, wherein the second compound targets at least one of the biomarkers selected from the group consisting of: interleukins, cytokines, and proinflammatory markers.

26. The method of claim 23, wherein the second compound is a CTLA4-Ig fusion protein, an abatacept, a belatacept, or a galiximab.

27. The method of claim 12, wherein the compound is administered orally, parenterally, or topically.

28. The method of claim 27, wherein the compound is administered parenterally.

29. The method of claim 28, wherein the compound is administered by injection, most preferably intravenous, intramuscular, or subcutaneous injection.

30. The method of claim 12, wherein the compound that targets CD40L or CD40 is an antibody that comprises:
(a) a heavy chain variable region (VH) comprising:
i) a CDRH1 domain comprising the sequence set forth in SEQ ID NO: 9;
ii) a CDRH2 domain comprising the sequence set forth in SEQ ID NO: 10, 11, 12, 13, or 14; and
iii) a CDRH3 domain comprising the sequence set forth in SEQ ID NO: 15; and
(b) a light chain variable region (VL) comprising:
i) a CDRL1 domain comprising the sequence set forth in SEQ ID NO: 16 or 17;
ii) a CDRL2 domain comprising the sequence set forth in SEQ ID NO: 18 or 19; and
iii) a CDRL3 domain comprising the sequence set forth in SEQ ID NO: 20.

31. The method of claim 12, wherein the compound that targets CD40L or CD40 is an antibody that comprises:
(a) a heavy chain variable region (VH) having the amino acid sequence as set forth in SEQ ID NOs: 1, 2, 3, or 4; and
(b) a light chain variable region (VL) having the amino acid sequence as set forth in SEQ ID NOs: 5, 6, 7, or 8.

32. A method of modulating the concentration of at least one biomarker in a subject, the method comprising:
administering to the subject a therapeutically effective amount of a compound against CD40L or CD40,
wherein the biomarkers are selected from the group consisting of: IgA, IgE, CD40L, CD40, C3, CXCL9, CXCL10, and IgM.

33. The method of claim 32, wherein the concentration of at least one biomarker increases following administration of the compound.

34. The method of claim 32, wherein the concentration of at least one biomarker decreases following administration of the compound.

35. The method of claim 32, wherein the concentration of at least one biomarker increases, and the concentration of at least one other biomarker decreases, following administration of the compound.

36. The method of claim 32, wherein the subject has a kidney-related disease or disorder.

37. The method of claim 36, wherein the kidney-related disease or disorder comprises inflammation.

38. The method of claim 32, wherein the subject is mammalian and/or human.

39. The method of claim 32, wherein the compound blocks the interaction between CD40 and CD40L.

40. The method of claim 32, wherein the compound is an anti-CD40L antibody or an anti-CD40 antibody.

41. The method of claim 32, wherein the compound is AT-1501.

42. The method of claim 32, wherein the compound is MR1 or 5c8.

43. The method of claim 36, wherein the kidney-related disease or disorder is colitis, drug induced lupus nephritis, lupus nephritis, nephropathy, chronic kidney disease, graft versus host disease, immune graft response, transplant rejection, atherosclerosis, diabetic kidney disease, acute kidney injury, kidney failure, polycystic kidney disease, hematuria, Fabry disease, lupus kidney disease, Alport's syndrome, IgM nephropathy, interstitial nephritis, Membranoproliferative Glomerulonephritis, simple kidney cysts, kidney infections, kidney stones, or IgA nephropathy.

44. The method of claim 36, wherein the kidney-related disease or disorder is an autoimmune disease, wherein the autoimmune disease is systemic lupus erythematous, type-1 diabetes, myasthenia gravis, inflammatory bowel disease, immune thrombocytopenic purpura, rheumatoid arthritis, psoriasis, Addison's disease, Crohn's disease, uveitis, multiple sclerosis, hemolytic anemia, or Graves' disease.

45. The method of claim 32, wherein the compound is administered at least once every three weeks.

46. The method of claim 32, wherein the compound is administered at a dose of between 200 mg/kg and 100 mg/kg; preferably at a dose between 1 mg/kg and 20 mg/kg.

47. The method of claim 32, wherein the compound is administered orally, parenterally, or topically.

48. The method of claim 47, wherein the compound is administered parenterally.

49. The method of claim 47, wherein the compound is administered by injection, most preferably intravenous, intramuscular, or subcutaneous injection.

50. The method of claim 32, wherein the compound that targets CD40L or CD40 is an antibody that comprises:
(a) a heavy chain variable region (VH) comprising:
i) a CDRH1 domain comprising the sequence set forth in SEQ ID NO: 9;
ii) a CDRH2 domain comprising the sequence set forth in SEQ ID NO: 10, 11, 12, 13, or 14; and
iii) a CDRH3 domain comprising the sequence set forth in SEQ ID NO: 15; and
(b) a light chain variable region (VL) comprising:
i) a CDRL1 domain comprising the sequence set forth in SEQ ID NO: 16 or 17;
ii) a CDRL2 domain comprising the sequence set forth in SEQ ID NO: 18 or 19; and
iii) a CDRL3 domain comprising the sequence set forth in SEQ ID NO: 20.

51. The method of claim 32, wherein the compound that targets CD40L or CD40 is an antibody that comprises:
(a) a heavy chain variable region (VH) having the amino acid sequence as set forth in SEQ ID NOs: 1, 2, 3, or 4; and
(b) a light chain variable region (VL) having the amino acid sequence as set forth in SEQ ID NOs: 5, 6, 7, or 8.
